(19) United States
(12) Patent Application Publication
(10) Pub. No.: US 2008/0108091 A1
(43) Pub. Date: May 8, 2008

(54) Proteomic Patterns of Cancer Prognostic and Predictive Signatures

(76) Inventors: Bryan T.J. Hennessy, Houston, TX (US); Gordon B. Mills, Houston, TX (US); Kevin Coombes, Houston, TX (US); Ana Gonzalez-Anguelo, Pearland, TX (US); Mark Carey, Houston, TX (US)

Correspondence Address: FULBRIGHT & JAWORSKI L.L.P., 600 CONGRESS AVE., SUITE 2400, AUSTIN, TX, 78701, US

(21) Appl. No.: 11/835,234

(22) Filed: Aug. 7, 2007

Related U.S. Application Data
(60) Provisional application No. 60/836,176, filed on Aug. 7, 2006.

(51) Int. Cl. G01N 33/574; C12N 5/06

(52) U.S. Cl. 435/7.23; 435/375

(57) ABSTRACT

The invention provides method for predicting whether a cancer patient will respond to a therapy. Methods of the invention may involve examining protein from a cell of the cancer patient by determining the binding of a panel of antibodies to the protein. Methods of the invention may be used to generate both expression and activation profiles for cells from a cancer patient. Profiles from a cancer patient may then be compared to known profiles for therapy responders and non-responders to predict the individual response of the patient. For example, methods of the invention may be used to determine whether an ovarian or breast cancer patient will respond to a therapeutic protocol.

This application claims priority to U.S. Provisional Patent application Ser. No. 60/836,176 filed Aug. 7, 2006, entitled “PROTEOMIC PATTERNS OF CANCER PROGNOSTIC AND PREDICTIVE SIGNATURES,” which is incorporated herein by reference in its entirety.

### BACKGROUND OF THE INVENTION

I. Field of the Invention

This invention relates to the use of a novel quantitative high throughput approach to characterize levels of proteins and their activation as continuous variables in cancer patient samples and/or cell lines. The patterns of protein expression and activation combined with quantitative or semi-quantitative analysis identify novel predictors of cancer behavior and response to therapy.

II. Background

Cancer remains a major health concern in the United Sates and world wide. For example, breast cancer is the second highest cause of cancer death in North American women (Pisani et al., 2002; Parkin et al., 2001). The breast cancer mortality rate in developing countries is even higher. Breast cancer exemplifies many types of cancer in that that it is a heterogeneous disease. Clinicopathologic criteria are used to guide therapy decisions, however this approach does not define tumor biology and tumors of the same grade and stage often behave very differently. As a result, a large percentage of patients treated with chemotherapy would not have relapsed, and thus receive needless toxic therapy, while a significant proportion of patients given therapy relapse anyway. To make more informed therapy decisions, a better understanding of the molecular mechanisms underlying the wide variation in cancer behavior is required.

In breast cancer for instance, hormone receptor status of breast cancer and other clinicopathologic factors have driven patient management for decades (Early Breast Cancer Trialists' Collaborative Group, 1998). More recently, several reports have described the use of transcriptional profiling to obtain a clinically relevant molecular classification of breast cancer (Sorlie et al., 2001). Breast cancer gene profiles can predict response to anthracyclines and taxanes (Ayers et al., 2004). The polymerase chain reaction-based Oncotype Dx (Genomic Health Inc.) can predict response to tamoxifen (Paik et al, 2004). However, these studies require validation and unfortunately using these algorithms, the positive and negative predictive values are not adequately optimal so as to allow truly individualized molecular therapy. In fact, although many individual proteins have been extensively studied as potential prognostic and predictive factors in breast cancer, only 3 are routinely accepted in current practice—estrogen receptor (ER), progesterone receptor (PR) and HER2/neu. However, several additional proteins have been found to correlate individually with some aspects of breast cancer behavior. Thus, the integrated study of the expression and activation of multiple proteins and signaling pathways may potentially provide a powerful breast cancer classifier and predictor. This approach may have utility on its own or may add to the power of assessment of gene expression changes.

Reverse Phase Protein Arrays (RPPAs) mRNA expression arrays have the ability to simultaneously measure the expression level of thousands of genes and identifies genomic subclasses that have advanced our understanding of breast cancer classification and to predict response to therapy (Sorlie et al., 2001; Ayers et al., 2004). However, comprehensive analysis of the transcriptome of cancer does not capture all levels of biological complexity. mRNA and protein levels are only roughly correlated and protein function is frequently uncoupled from mRNA levels. It is likely that important additional information resides at the protein level and in particular at the level of protein function (Gygi et al., 1999; Diks and Peppelenbosch, 2004). Furthermore, protein levels and function depend not only on translation but also on post translational modifications such as phosphorylation, prenylation, and glycosylation. As proteins are the major effectors of genomic information and changes as well as the direct mediators of cellular function, functional proteomic analysis has the potential to characterize cellular and cancer behavior as well as, if not better than, transcriptional profiling. Traditional protein assay techniques like Western blotting (WB) can assess the expression and phosphorylation of only a limited number of proteins. Additional methods of assessing levels and activation status (e.g., phosphorylation) of proteins in cancer cells are needed.

### SUMMARY OF THE INVENTION

Reverse phase protein microarrays (RPPAs) offer a new method to conduct comprehensive quantitative profiling of levels and activation status (e.g., phosphorylation) of many proteins in cancer cells (Charboneau et al., 2002). RPPAs can map intracellular signal transduction, proliferation, and apoptotic pathways in a comprehensive, convenient and sensitive manner (Charboneau et al., 2002). Since RPPAs can assay the total levels of a large number of proteins and their active (e.g., phosphorylated) forms, this technology may more accurately reflect pathogenic cellular molecular machinery than gene profiling. Potent clinical uses of RPPAs are being explored (Wulfkuhle et al., 2003; Grubb et al., 2003). However, to date their have not been methods described for using RPPA to predict the prognosis of a cancer patient or the propensity for response to a therapy. Prognosis is a medical term denoting how a patient's disease will progress and whether there is a chance for recovery. Whereas, a propensity for response to therapy is a prediction or assessment of the success of a treatment and is not necessarily related to prognosis.

In certain embodiments the present invention provides methods for evaluating a cancer patient. In certain aspects, the methods include predicting a cancer patient's (i.e., propensity) response to a therapy by examining proteins in the cells of the cancer patient. Typically, a sample obtained from the patient will contain at least one or more cancer cells. Such a method may comprise subjecting (e.g., contacting) proteins of the caner patient's cells to an antibody panel, i.e., two or more antibodies, under binding conditions and assessing the binding of the antibodies to the proteins. An assessment of the binding of the proteins and antibodies binding can be used to generate a profile that can then be compared to a known profile for a therapy responder or non-responder. Thus, a comparison of the profiles can be used to predict the patient's response or propensity for response to a therapy or the lack thereof. In certain aspects the comparison of profile is used to evaluate the propensity of a patient to be effectively treated by a therapy or combinations of therapies. In another aspect, a detrimental therapy may be identified so that a treating physician can choose an alternative therapy or minimize the detrimental effects of a selected therapy. In some specific cases, methods of the invention may be used to predict the probability that a cancer patient will respond or will not respond to a therapy at a level sufficient for a therapeutic benefit. A therapeutic benefit includes, but is not limited to reduction or cessation of growth of a tumor or cancer; relief, mitigation, or palliation of a condition directly or indirectly resulting from a tumor or cancer, a killing or growth cessation of all or part of a tumor or cancer, and other measures of therapeutic benefit recognized in the art.

As used herein the phrases “panel of antibodies” or “antibody panel” refer to a set of antibodies that bind to a plurality of different cellular targets or proteins. For example, a panel of antibodies may bind to at least 2, 3, 4, 5, 6, 7, 8, 9, 10 15, 20, 25, 50 or more cellular targets, proteins, and/or protein modifications, including all values and ranges there between. In a preferred embodiment, at least one antibody in a panel is an antibody that binds preferentially to a protein that comprises a posttranslational modification. A skilled artisan will recognize that the term post-translational modification comprises a number of covalent protein modifications that have important regulatory functions, such as protein phosphorylation, methylation, acetylation, glycosylation, myristoylation, prenylation, and/or protein ligation (e.g., ubiqutination, sumylation or NEDDylation of proteins). Furthermore, post-translational modifications may also refer to protein cleavage. Thus, in certain aspects, an antibody panel comprises at least one phosphorylation, methylation, acetylation, gylcosylation, myristoylation, prenylation, ubiquitination, sumylation, NEDDylation or proteinase cleavage product specific antibody. Such a post-translational modification specific antibody will preferentially bind (e.g., bind at a detectably higher level to one form of a protein as compared to another form) to a protein that comprises or does not comprise a particular posttranslational modification (e.g., a phosphorylated protein).

Thus, it will be understood that in certain aspects the invention provides a method for predicting a cancer patient's response to a therapy and/or a patient's propensity to sufficiently benefit from a therapy by examining protein expression or activation in the cells of the cancer patient. In some embodiments, examining protein may comprise quantifying or estimating the amount of a protein, activated protein, or inactivated protein, and/or detecting the presence or absence of a protein or protein modification at a certain level. As used herein the term “activated protein” means a protein that is functionally active. For example, an activated kinase phosphorylates target molecules and activated transcription factors mediated transcription at target promoters. In some aspects, an activated protein may be a protein that comprises or does not comprise a specific post-translational modification (e.g., phosphorylation may deactivate certain proteins). The term protein expression as used here refers to the amount of a protein in a cell or population of cells.

Quantifying or estimating expression or activation of protein according to the invention may be a relative quantification, for example comparing the expression or activation in patient sample to expression or activation in a known sample or reference (e.g., digital or standard reference profile). In still further cases, quantifying protein in a sample (e.g., activated or inactivated protein) may comprise determining the concentration of a protein. In other aspects, the proportion of modified protein in a sample compared to unmodified protein can be determined. For example, in some aspects protein from a cell may be examined at a two more dilutions in order to more accurately quantify the amount of a protein. It will further be understood that a comparison between a patient sample or profile and a know sample or profile may be normalized by comparing about an equal number of cells, an equal mass of protein or an equal number of a particular protein known to have a approximately equal expression in a number of cell types.

It will also be understood by the skilled artisan that assessing the binding of an antibody in the methods of the invention may be by detection of a label. In certain cases, an antibody or panel of antibodies may be labeled, however in certain cases proteins from the cells of a patient may labeled. Labels for use in the invention include but are not limited to enzymes, radio isotopes, fluorescent labels, and luminescent labels. Thus, in certain cases detecting the binding of an antibody will involve immobilizing either the antibody and/or protein from the cells of a patient. In some aspects of the invention, cell proteins may be immobilized within an array, such as solid support may be made of nitrocellulose or a nitrocellulose coated support, and then labeled antibodies are bound to the protein and detected. In yet a further aspect, methods according to the invention may be automated. For example, robotic devices may be used to deposit spots of cell proteins or antibodies onto an array and/or computers may be used to compare binding profiles, such as a target, responder, and/or non-responder profiles.

In certain embodiments an antibody panel of the invention comprises at least one antibody that binds to a hormone receptor or growth factor receptor protein. For example, a panel may comprise an antibody that binds to an estrogen receptor (e.g., estrogen receptor alpha) and/or progesterone receptor. In another example, an antibody panel may comprise an antibody that binds to epidermal growth factor receptor (EGFR). Furthermore, in some cases an antibody panel may comprise antibodies that bind to two or more proteins in growth factor receptor signaling pathway. In the case of the epidermal growth factor receptor (EGFR)/HER2/phosphatidylinositol 3-kinase(PI3K)/AKT pathway for instance antibody panels comprising multiple pathway member binding antibodies may be advantageous since multiple mutations in the PI3K pathway are present in certain cancers (e.g., breast cancer) (Stoica et al., 2003; Bachman et al., 2004). In certain aspects, since activating mutations in PI3K itself are common mutations in cancer at least one antibody that binds to activated PI3K may included in an antibody panel of the invention.

In still a further embodiment, and antibody panel of the invention may bind to at least one kinase protein. For example, an antibody panel of the invention may comprise at least one antibody to a Janus kinase (JAK), Mitogen activated protein kinase (MAPK), ERK1/2, MNK 1/2, S6 kinase, Akt, p38, mTor, PI3K, PKC, ras, b-raf or JNK. Furthermore, in preferred aspects of the invention the kinase binding antibody may be a phosphorylation specific antibody. In a specific example, an antibody panel of the invention comprises one or more antibody that binds to a protein or activated protein in the MAPK/ERK1/2 pathway. Some breast cancers have high levels of MAPK signaling, despite relatively infrequent mutation of RAS or b-RAF. Dual blockade of EGFR and ERK1/2 phosphorylation increases growth inhibition. MAPK pathway activation can bypass inhibition of EGFR/HER2 and may lead to chemotherapy resistance, thus detection of activated MAPK may be used predict therapeutic responsiveness.

In yet further embodiments an antibody array of the invention may be defined as an antibody array comprising antibodies that bind to at least 1, 2, 3, 4, 5 or more proteins in the Her2, PI3K, MAPK or STAT signaling pathways. In certain specific cases, an antibody panel of the invention comprises an E cadherin, PKC, p27, Cyclin B1 or p53 binding antibody. In some additional cases an antibody array or panel of the invention may comprise a Glutathione-S-transferase (GST), topoisomerase IIα (TOPO), survivin and/or tau binding antibody. These proteins have all been implicated in the responsiveness of breast tumors to chemotherapy (Paik et al., 2004; Murthy et al., 2005; Pusztai et al., 2004). Furthermore, they are differentially expressed in breast tumors and amplification of GST, often in ER-positive tumors, may lead to chemo resistance while amplification of TOPO may increase chemotherapy responsiveness.

In certain aspects, an antibody panel according to the invention may comprise antibodies that bind to estrogen receptor, E cadherin, phosphorylated Akt, phosphorylated MAPK, phosphorylated JNK and/or phosphorylated S6. Such a panel of antibodies may be used to predict an ovarian cancer patient's response to a therapy. In another embodiment and antibody panel may comprise antibodies that bind to estrogen receptor, phosphorylated p38 and p53. In some cases such a panel may be used according to the invention to predict a breast caner patient's response to a therapy.

In other aspects, the antibodies can be selected from E cadherin, 4 EBP, PKC, p53, estrogen receptor, progesterone receptor, S6, AKT, Her2, Src, PI3K, p38, p27, mTOR, JNK, MAPK (44/42), cyclin D1, and/or cyclin B1.

In still further aspects, the antibodies bind at least ER and p38.

In yet further aspects, the antibodies bind at least ER, PR, AKT, p38, and mTOR.

In certain aspects, the antibodies bind at least two of ER, E cadherin, AKT, MAPK (44/42), C-jun N-Terminal kinase (JNK), or S6.

In a further aspect, the antibodies bind at least ER, E cadherin, AKT, MAPK (44/42), C-jun N-Terminal kinase (JNK), and S6.

In still a further aspect, the antibodies bind at least src, AKT, HER2, S6, and cyclin D1.

In certain embodiments, a group of antibodies may have a predictive value of 75, 80, 85, 90, 95, 98, or 99%, including all values and variables there between, in predicting a tumor is susceptible or resistant to a particular therapy.

In certain aspects of the invention methods may involve treating cells from a patient with a growth or proliferation stimulator or inhibitor prior to examining the proteins from the cell. In certain cases, treatment with such a stimulator or inhibitor performed on cells in tissue culture (in vitro or ex vivo) or on cells that are still in a patient (in vivo). For example, preoperative (neoadjuvant) chemotherapy (PC) downstages tumors and permits in vivo assessment of tumor response (e.g., via methods of the invention) providing an opportunity to predict outcome, evaluate biological marker expression, and tailor therapy (Fisher et al., 2002). In certain cases, methods of the invention may be used to determine if a particular therapy is effective for a patient or optimally results in pathological complete response (pCR) which is associated with an excellent long-term prognosis. For instance some stimulators and inhibitors for use in methods of the invention include but are not limited to cancer as well insulin-like growth factor (IGF), fibroblast growth factor (FGF), epithelial growth factor (EGF), platelet derived growth factor (PDGF), hormones (e.g., estrogen), trastuzumab, tyrosine kinase inhibitors, PI3K inhibitors, as well as any other chemotherapeutic or immunotherapeutic molecules.

The skilled artisan will understand that growth stimulators or inhibitors will agonize or antagonize at least one cellular signaling pathway. Thus, it will be understood that in some embodiments methods of the invention may involve agonizing or antagonizing a signaling pathway in a cell from a patient and then examining the proteins of the cell by subjecting the proteins of the cell to a panel of antibodies. In some embodiments, a panel of antibodies for use in this aspect of the invention may comprise antibodies that bind to one, two or more proteins in the signaling pathway that is being agonized or antagonized.

In certain aspects of invention the cancer patient may be a lung, breast, brain, prostate, spleen, pancreatic, cervical, ovarian, head and neck, esophageal, liver, skin, kidney, leukemia, bone, testicular, colon, or bladder cancer patient. For instance, in a preferred embodiment the cancer patient is an ovarian or breast cancer patient. It will be understood that cells from a cancer patient maybe comprised in a sample from the cancer patient. In some embodiments, the cells may be cancer cells, for instance such as cells comprised in a tumor biopsy sample. However, in certain other cases the cells will not comprise cancer cells, for example a cell sample may be a sample of tissue surrounding a tumor, a blood sample or a cheek swab.

As used herein the term therapy refers to any therapy administered or to be administered to a cancer patient. For example, the therapy may be a chemotherapy, a radiation therapy, an immunotherapy, or a surgical therapy. In certain embodiments the therapy is chemotherapy. In a further embodiment the therapy is radiation therapy. In still further embodiments the therapy is immunotherapy. Chemotherapies according to the invention include but are not limited to a cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, famesyl-protein transferase inhibitors, transplatinum, 5-fluorouracil, vincristin, Velcade, vinblastin or methotrexate therapy. Immunotherapies of the invention may include administration of antibodies that target hormone receptors, angiogenic factors, or cancer cell markers, e.g., Herceptin, Avastin, or a cancer cell targeted immunotoxins.

In still yet a further embodiment, there is provided a kit for predicting a cancer patient's response to a therapy and/or propensity to benefit from a course of treatment. Such a kit may comprise one or more of a panel of antibodies, a composition for detecting antibody binding to proteins, a responder or non-responder antibody binding profile (e.g., a reference array or a digital reference of either or both), a microarray slide, a protein extraction buffer, a cell proliferation inhibitor, a cell proliferation stimulator, and/or a computer program for comparing antibody binding profiles. In certain cases, such a kit may be comprised in a convenient enclosure such as a box. Furthermore, a kit of the invention may include instructions for use of the reagents therein.

Embodiments discussed in the context of a methods and/or composition of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: An example of a protocol for printing cell protein lysates onto nitrocellulose micro array slides.

FIGS. 2A-2B: An example of data from a reverse phase protein array (RPPA). FIG. 2A, shows the dilutions of cell lysate or protein standard used in the analysis. FIG. 2B, dilutions samples for a protein standard (top two rows) or tissue culture cells that are either stimulated or unstimulated (as indicated across the bottom) are printed on the array. The array is probed with a monospecific antibody that binds to phosphorylated AKT (AKT(S473)). The amount of protein in the standard for each dilution is shown.

FIGS. 3A-3D: Validation of the RPPA assay methods. FIG. 3A, spots comprising the same protein samples reliably indicate the same amount of protein in the sample. FIG. 3B, HER2 protein assessed by RPPA (y-axis) correlates with HER2 gene copy number (x-axis), p<0.0001. FIG. 3C, ER protein assessed by RPPA (y-axis) correlates with transcription profiling of ER expression (x-axis), p<0.0001. FIG. 3D, PTEN protein assessed by RPPA (y-axis) correlates with transcription profiling of PTEN expression (x-axis), p<0.001.

FIG. 4: ‘Supervised’ outcome predictor: 44 stage III/IV high-grade ovarian cancer patient test samples were assayed by RPPA using antibodies to ER, E cadherin, phosphorylated AKT, phosphorylated S6, phosphorylated JNK and phosphorylated MAPK. “>” indicates suboptimal tumor debulking while “<” indicates optimal debulking.

FIG. 5: ‘Supervised’ outcome predictor from 44 patient test set applied to 28 high-grade ovarian cancer patient validation set. Antibodies for RPPA are as indicated for FIG. 4. “>” indicates suboptimal tumor debulking while “<” indicates optimal debulking.

FIG. 6: Predictive RPPA signature for relapse in patients with adjuvant antihormone-treated high-grade early stage hormone receptor-positive breast cancer. The components of this particular signature that were derived from Table 1 are p70S6 Kinase, stat 3, MEK1(p)Ser217/221, p38, p38(p)Thr180/Tyr182 and S6(p)Ser235-236. On clustering, two main subgroups were identified (called clusters 1 and 2) with significantly different outcomes (all relapses/stage IV cases after adjuvant anti-hormone therapy occurred in group 1). The survival plot demonstrates relapse-free survival.

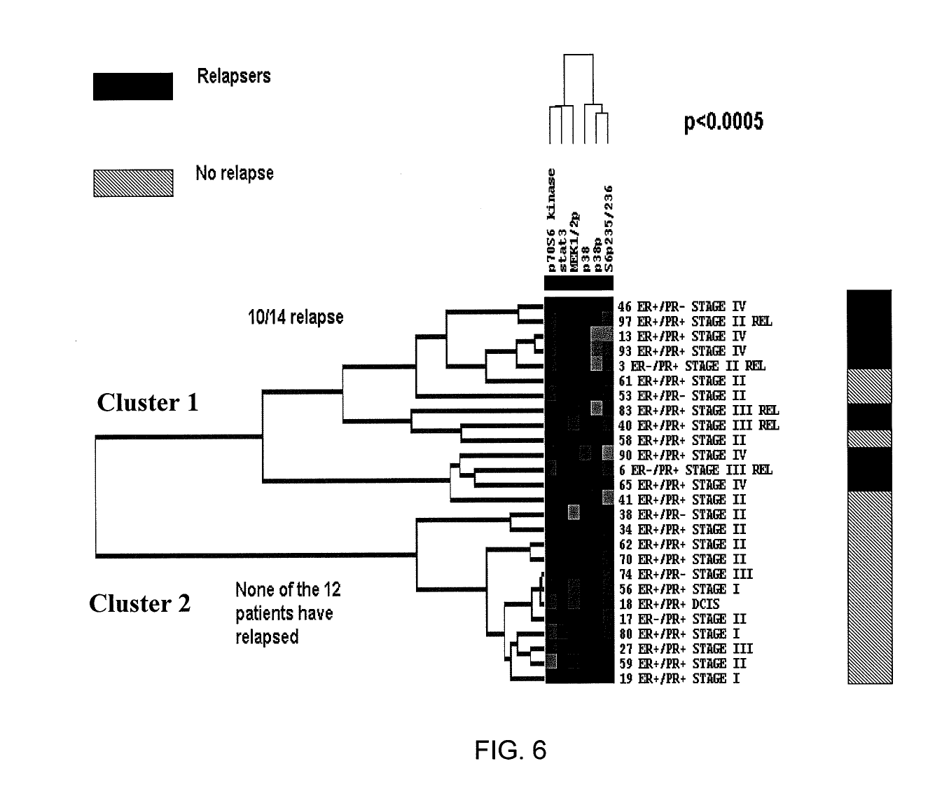

FIG. 7: Shows results from an RPPA employing antibodies that bind to phosphorylated mTor, phosphorylated p38 ER, PR, and phosphorylated Akt. The RPPA accurately predicts 6/6 relapses post adjuvant hormonal therapy.

FIG. 8: Shows results from an RPPA employing antibodies that bind to phosphorylated mTor, phosphorylated p38 ER, PR, and phosphorylated Akt. The RPPA accurately predicts 5/5 stage IV disease post adjuvant hormonal therapy.

FIG. 9: Shows the result of an RPPA using antibodies that bind to ER and phosphorylated AKT (phosphorylation/activation at Serine 473). Relapse cases indicated by the black bar to the right of the figure.

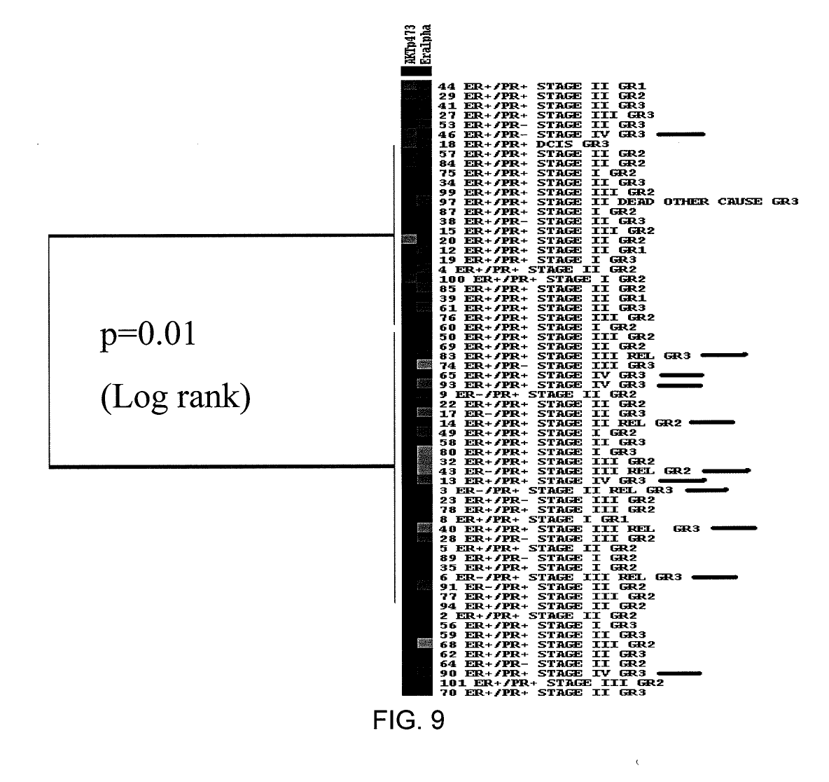

FIG. 10: Activation of the membrane receptor tyrosine kinase (RTK) and phosphatidylinositol-3-kinase (PI3K)/AKT pathways is associated with low tumor estrogen receptor (ER) expression and poor outcomes of patients with epithelial ovarian cancer (EOC) after standard primary platinum-based chemotherapy. Reverse phase protein lysate array (RPPA) was used to quantify and integrate the expression of ER, EGFR and src with activation (i.e., phosphorylation) of protein kinase C (PKC) alpha (PKCα(p)657), AKT (AKT(p)Ser473), glycogen synthase kinase (GSK) 3 (GSK3(p)Ser21/9) and ribosomal S6 protein (S6(p)Ser240/244) to form a prognostic RTK-PI3K/AKT pathway activation signature. The signature components are EGFR, ER, src, AKT, GSK3, PKCα(p)657, AKT(p)Ser473, GSK3(p)Ser2i/9 and S6(p)Ser240/244. On unsupervised clustering, two main subgroups were identified (called ER and Akt) with significantly different outcomes. The prognostic ability of this signature is independent of stage and grade on multivariable analysis.

FIG. 11: Even unsupervised clustering approaches can distinguish epithelial ovarian cancer (EOC) subsets with significantly different survival outcomes, pointing to the obvious importance of the antibodies in Table 1 below to the clinical behavior of EOC. O indicates the percentage of EOCs in each group or cluster that progress at a time shorter than the recognized median progression-free survival (PFS) time of 15.5 months for EOC after standard paclitaxel/carboplatin primary chemotherapy in large prospective clinical trials.

FIG. 12: Activation of the membrane receptor tyrosine kinase (RTK) and phosphatidylinositol-3-kinase (PI3K)/AKT pathways is associated with low estrogen receptor (ER) expression and poor outcomes of 65 patients with early stage hormone receptor-positive breast cancer after treatment with standard adjuvant antihormone therapy. Reverse phase protein lysate array (RPPA) was used to quantify and integrate the expression of ER, EGFR and src with the activation (i.e., phosphorylation) of protein kinase C (PKC) alpha, AKT, glycogen synthase kinase (GSK) 3 and ribosomal S6 protein to form a prognostic RTK-PI3K/AKT pathway activation signature. The signature components are EGFR, ER, src, AKT, GSK3, PKCα(p)657, AKT(p)Ser473, GSK3(p)Ser21/9 and S6(p)Ser240/244. On unsupervised clustering, two main subgroups were identified (called ER and PI3K) with significantly different outcomes. The prognostic ability of this signature is independent of stage and grade on multivariable analysis. The survival plot demonstrates relapse-free survival.

FIGS. 13A-13B: When reverse phase protein array (RPPA) is used to quantify only ER expression and Akt phosphorylation in early-stage hormone receptor-positive breast cancer, the breast cancer signature retains significant predictive capability after adjuvant antihormone therapy. This is shown in FIG. 13A utilizing AKT phosphorylation at Serine 473 (AKT(p)Ser473 as a surrogate for PI3K pathway activation) and ERα level (p=0.02 for significant inverse correlation). This signature of low (=green) ERα expression with high (=red) AKT(p)Ser473 provides strong prediction of disease recurrence after adjuvant antihormone therapy (relapses marked by the transecting line in FIG. 13A; Kaplan-Meier curve shown in FIG. 13B (p=0.04)).

FIG. 14: Analysis of hormone receptor positive breast cancer reverse phase protein array data by resampling analysis using pearson correlation, linear discriminant analysis (LDA) and K nearest neighbours (KNN) methodology to determine (phospho)proteins associated with breast cancer relapse after adjuvant antihormone therapy. The signature components are those shown in the table below. The following are the antibodies that resulted in high sensitivities (>0.8), ordered by frequency.

FIG. 15: RPPA signature that we have preliminarily validated in adjuvant antihormone-treated patients with early stage hormone receptor-positive breast cancer. The signature components of this particular predictive signature that were derived from Table 1 are ER, PR, p38(p)Thr180/Tyr182, Akt(p)Thr308 and mTOR(p)Ser2448. On unsupervised clustering, two main subgroups were identified in both tumor sets (called 1 and 2) with significantly different outcomes (all relapses/stage IV cases after adjuvant anti-hormone therapy occurred in group 1 in each case). The prognostic ability of this signature is independent of stage and grade on multivariable analysis.

FIG. 16: Predictive RPPA signature for relapse in patients with adjuvant cytotoxic chemotherapy-treated triple receptor-negative breast cancer. The components of this particular signature that were derived from Table 1 are p7OS6K(p)Thr389, FKHRL1, FKHRL1(p)Ser318/321 and S6(p)Ser240-244. On clustering, two main subgroups were identified (called Groups A and B) with significantly different outcomes (all relapses/stage IV cases after adjuvant cytotoxic chemotherapy occur in Group B). The survival plot demonstrates relapse-free survival.

FIG. 17: Activation of the membrane receptor tyrosine kinase (RTK) and phosphatidylinositol-3-kinase (PI3K)/AKT pathways is associated with low estrogen receptor (ER) expression in early stage hormone receptor-positive breast cancer. However, this signature is not prognostic when patients are not treated with adjuvant antihormone therapy. Reverse phase protein lysate array (RPPA) was used to quantify and integrate the expression of ER, EGFR and src with the activation (i.e. phosphorylation) of protein kinase C (PKC) alpha, AKT, glycogen synthase kinase (GSK) 3 and ribosomal S6 protein to form the PI3K/AKT pathway activation signature as in FIGS. 2 and 4. The signature components are EGFR, ER, src, AKT, GSK3, PKCα (p)657, AKT(p)Ser473, GSK3(p)Ser21/9 and S6(p)Ser240/244. On unsupervised clustering, two main subgroups were identified (called ER and PI3K). The survival plot demonstrates relapse-free survival.

FIG. 18: Analysis of reverse phase protein array data by resampling analysis using pearson correlation, linear discriminant analysis (LDA) and K nearest neighbours (KNN) methodology to determine (phospho)proteins associated with early stage hormone receptor-positive breast cancer relapse after no adjuvant therapy. The signature components are those shown in the table below. The following are the antibodies that resulted in high sensitivities (>0.8), ordered by frequency.

FIG. 19: A striking inverse association between ER expression and PI3K/AKT/mTOR pathway activation (specifically between ER expression (on the right of each heat map below) and Akt(p)Ser473 (on the left of each heat map below)) has been consistently seen in our breast cancer and epithelial ovarian cancer (EOC) tumor set RPPA data. In each case, the correlation coefficient (CC) corresponds to a p value of <0.05. These data suggest an important association and the underlying mechanisms therefore need exploration.

FIG. 20: Functional proteomic signature for PIK3CA mutation derived using reverse phase protein array quantitation data for (phospho)proteins shown in Table 1. Heat maps in hormone receptor-positive (ER+) breast cancer cell lines and human tumors were constructed. This signature detects PIK3CA-mutant cell lines and human tumors with the sensitivities and specificities shown. The PIK3CA mutation signature (b) was associated with a trend (p=0.06) towards improved patient relapse-free survival (RFS) compared with the PTEN signature (a) after adjuvant antihormone treatment for early stage hormone receptor-positive breast cancer.

### DETAILED DESCRIPTION OF THE INVENTION

The invention concerns cancer prognostic and predictive signatures developed using quantification of the expression and/or activation of cellular proteins, for example using reverse phase tissue lysate array-based methods. For instance, activation and expression of protein kinases (e.g., phosphatidylinositol-3-kinase (PI3K)/Akt and mitogen activated protein kinase (MAPK) for breast cancer) and steroid signaling pathways may be determined by methods of the invention and used to predict a clinical outcome for patients. Thus, signatures may be useful as a guide to patient prognosis and also for prediction of the likelihood (propensity) that individuals with specific cancer subtypes will derive benefit from specific therapies (hormonal therapy, chemotherapy, and targeted therapy (trastuzumab)). Consistent with the latter use, the invention can be used to identify protein signatures indicative of individual patient requirements for therapeutic strategies to overcome treatment (e.g., antihormone) resistance.

Various array and profiling methodologies (e.g., transcriptional profiling, comparative genomic hybridization) are currently being explored in an attempt to improve prediction of prognosis of and likelihood of benefit for individual patients with specific breast cancer subtypes after treatment with appropriate therapy as specified by widely accepted clinicopathologic variables (e.g., hormonal therapy in those with hormone receptor-positive breast cancer). However, in spite of multiple studies, the only test approved to further stratify patients with a specific breast cancer subtype (in this case hormone receptor-positive) to treatment based on their potential to benefit from specific therapies is Oncotype Dx (Paik et al., 2004)). However, many current methodologies assay DNA and mRNA levels and are not capable of providing information on the expression and activation of proteins, which are the direct mediators of cell behavior. The approach described herein employs a new proteomic technology capable of quantifying not only protein expression levels but also protein activation status. Reverse phase tissue lysate arrays (i.e., reverse phase protein arrays (RPPA)) quantify protein expression and activation and may thus be more useful than genomic and transcriptional technologies in predicting probable behavior of individual tumors, particularly when the proteins assayed or their encoding genes or mRNAs are already implicated by other studies in carcinogenesis. In addition, lysate arrays are one of the most sensitive protein detection technologies developed to date and are capable of determining activation of cellular proteins present in the femtogram range. RPPAs are high-throughput and can easily, efficiently, and simultaneously assay the levels of hundreds of proteins in a multitude of tumor samples.

For example, only about 60% of hormone receptor positive tumors respond to hormonal modulation. ER protein levels using tissue lysate arrays correlate inversely with the amount of Akt phosphorylation in hormone receptor positive breast tumors. Other data indicates that the PI3K/Akt and MAPK pathways may activate ER in a hormonally independent manner through receptor phosphorylation. Since hormonal manipulation only blocks hormone dependent ER activation and since studies herein may indicate that the quantity of ER protein is the major driver of outcome after antihormonal therapy, this suggests that tissue lysate array-based approach may be capable of stratifying patients with hormone receptor positive breast cancer to a treatment decision based on quantification of ER and activation status of various components of kinase signaling pathways.

### I. Reverse Phase Protein Array (RPPA)

In certain embodiments, tissue or cellular lysates can be obtained by mixing tissue sample material with lysis buffer and then serially diluted (e.g., 8 serial dilutions: full strength, ½, ¼, ⅛, 1/16, 1/32, 1/64, 1/128) with additional lysis buffer. Dilutions can be made with Tecan liquid handling robot or other similar devices. This material can printed/spotted onto a substrate, such as nitrocellulose-coated glass slides (FAST Slides, Schleicher & Schuell BioScience, Inc. USA, Keene, N.H.) with an automated GeneTac arrayer (Genomic Solutions, Inc., Ann Arbor, Mich.) or other similar devices. In certain aspects, as many as 80 samples can be spotted in 8 serial dilutions on a single substrate. Serial dilutions can provide a slope and intercept allowing relative quantification of individual proteins. Typically, measurements of protein are compared to control peptides allowing absolute quantification.

Typically, after slide printing, the same stringent conditions for slide blocking, blotting and antibody incubation used for western blotting are applied prior to the addition of the primary antibody. The DAKO (Copenhagen, Denmark) signal amplification system can be used to detect and amplify antibody-binding intensity. Signal intensity is measured by scanning the slides and quantifying with software, such as the MicroVigene automated RPPA software (VigeneTech Inc., Massachusetts), to generate sigmoidal signal intensity-concentration curves for each sample. To accurately determine absolute protein concentrations, standard signal intensity-concentration curves for purified proteins/recombinant peptides of known concentration are generated for comparison with the samples in which protein concentrations are unknown. The RPPAs are quantitative, sensitive, and reproducible. RPPA may also be validated with mTOR, erk, p38, GSK3 and JNK as stable loading controls.

Quantified protein expression data is analyzed, using programs and algorithms identical to those used for analysis of gene expression arrays. The data is analyzed for the presence of clusters based on differential protein expression using methods available, for example, in the R statistical software package (cran.r-project.org). A variety of clustering methods (including hierarchical clustering, K-means, independent component analysis, mutual information, and gene shaving) are used to classify samples into statistically similar groups. For example, Xcluster (SMD software, Paulo Alto, Calif.) and TreeView (University of Glasgow, Glasgow, Scotland) software may be used to put all this data together into unsupervised hierarchical clusters or heat maps which arrange the samples in terms of similarity in protein expression and activation. Robustness and statistical significance of these groups may be evaluated by bootstrap data resampling (Kerr and Churchill, 2001). In addition to primary clustering analysis based on all proteins, secondary bootstrap-resampled clustering analyses may be performed using proteins in a signaling pathway of interest.

Typically, for a cluster that is statistically significant based on bootstrap resampling to represent an important subtype of breast cancer, the cluster should contain samples from at least 5 patients. For instance, using the 80 samples a breast cancer subtype with 10% prevalence will have a 90% probability of contributing at least 5 samples to the study population. Thus, the proposed patient sample should be sufficient to detect subtypes with at least 10% prevalence. A potential problem is batch effect since analyses are performed on more slides than can be printed at one time. However, evidence suggests that inter-slide variation is minimal (R2>0.8) when slides are printed at different times and stained with the same antibody. As new, potentially relevant proteins are identified stored sample preparations/plates may be used to probe for these novel proteins and the data can be incorporated into the dataset for analysis. Thus, the sample set will be continuously enriched. As only a small amount of lysate is required, the samples can accommodate analysis of up to a thousand antibodies easily.

Patient samples are typically linked to an oncology database such as the Breast Medical Oncology Database, which includes patient characteristics and outcome information (response to PC, type of therapy, etc.). These data can be correlated with the RPPA clusters using standard statistical methods, including Fisher's exact test, analysis of variance, and Cox proportional hazards models for time to recurrence. In this way, it can be determined if clusters of patient samples generated by RPPAs have clinical significance and correlate with a specific endpoint: e.g., pathological complete response (pCR).

Supervised statistical approaches may also be employed to assist in building a pCR predictor. Adequate power to determine differences will require a ‘training set’ (e.g., 80 samples). In addition, the inventors contemplate identifying kinase signaling patterns in chemotherapy-unresponsive tumors that can be targeted to augment the efficacy of cytotoxic treatment.

### II. Proteins, Cells And Cell Samples

It will be understood by one of skill in the art that in order to assess the binding of proteins from a cancer patient to a panel of antibodies a sample of protein from the patient will be examined. In certain aspects of the invention, methods for obtaining such as sample are included as part of the invention. However, in other aspects of the invention the proteins for method of the invention may be obtained from samples that have already been collected, such as frozen tissue, blood, or biopsy samples.

In a certain embodiments of the invention, proteins from cells of a cancer patient are analyzed. Such cells may be from any part of the patient for example the cells may be from the bladder, blood, bone, bone marrow, brain, breast, colon, esophagus, gastrointestine, gum, head, kidney, liver, lung, nasopharynx, neck, ovary, prostate, skin, stomach, testis, tongue, uterus or other tissue or organ sample. In certain specific cases the cells from the cancer patient may be cancer cells. Some cancer cells that may be used according to the invention include but are not limited to: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### III. Antibodies And Methods For Their Production

As described above certain aspects of the invention involve the use of antibodies. Antibodies can be made by any of the methods that as well known to those of skill in the art. In certain embodiments an antibody recognizes a covalently modified protein, such a phosphorylated protein. The following methods exemplify some of the most common antibody production methods.

### A. Polyclonal Antibodies

Polyclonal antibodies generally are raised in animals by multiple subcutaneous (sc) or intraperitoneal (ip) injections of the antigen. As used herein the term “antigen” refers to any polypeptide that will be used in the production of antibodies. However, it will be understood by one of skill in the art that in many cases antigens comprise more material that merely a single polypeptide. In certain other aspects of the invention, antibodies will be generated against specific polypeptide antigens. In some cases the full length polypeptide sequences may be used as an antigen however in certain cases fragments of a polypeptide (i.e., peptides) may used. In still further cases, antigens may be defined as comprising or as not comprising certain post translational modifications such, phosphorylated, acetylated, methylated, glycosylated, prenylated, ubiqutinated, sumoylated or NEDDylated residues. In another example, antibodies can be made against polypeptides that have been identified to be expressed on the surface of cancer cells, such as Her-2. Thus one of skill it the art would easily be able to generate an antibody that binds to any particular cell or polypeptide of interest using method that are well known in the art.

In the case where an antibody is to be generated that binds to a particular polypeptide it may be useful to conjugate the antigen or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹ N═C═NR, where R and R¹ are different alkyl groups.

Animals are immunized against the immunogenic conjugates or derivatives by, for example, combining 1 mg or 1 μg of conjugate (for rabbits or mice, respectively) with 3 volumes of Freud's complete adjuvant and injecting the solution intradermally at multiple sites. One month later the animals are boosted with about ⅕ to 1/10 the original amount of conjugate in Freud's complete adjuvant by subcutaneous injection at multiple sites. Seven to 14 days later the animals are bled and the serum is assayed for specific antibody titer. Animals are boosted until the titer plateaus. Preferably, the animal is boosted with the same antigen conjugate, but conjugated to a different protein and/or through a different cross-linking reagent. Conjugates also can be made in recombinant cell culture as protein fusions. Also, aggregating agents, such as alum, or other adjuvants may be used to enhance the immune response.

### B. Monoclonal Antibodies

In further embodiments of the invention, the cell targeting moiety is a monoclonal antibody. By using monoclonal antibodies cell targeting constructs of the invention can have greater specificity for a target antigen than targeting moieties that employ polyclonal antibodies. Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, monoclonal antibodies of the invention may be made using the hybridoma method first described by Kohler & Milstein (1975), or may be made by recombinant DNA methods (U.S. Pat. No. 4,816,567).

In the hybridoma method, a mouse or other appropriate host animal is immunized as described above to elicit lymphocytes (i.e., plasma cells) that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding 1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the target antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson & Pollard (1980).

After hybridoma cells are identified that produce antibodies of the desired specificity (e.g., specificity for a phosphorylated vs. un-phosphorylated antigen), affinity, and/or activity, the clones may be subcloned by limiting dilution procedures and grown by standard methods, Goding (1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention may be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. (1984), or by covalently joining to the immunoglobulin coding sequence all or part of the coding sequence for a non-immunoglobulin polypeptide. In that manner, “chimeric” or “hybrid” antibodies are prepared that have the binding specificity for any particular antigen described herein.

Typically, such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for the target antigen and another antigen-combining site having specificity for a different antigen. Chimeric or hybrid antibodies also may be prepared in vitro using known methods in synthetic protein chemistry.

For some applications, the antibodies of the invention will be labeled with a detectable moiety. The detectable moiety can be any one which is capable of producing, either directly or indirectly, a detectable signal. For example, the detectable moiety may be a radioisotope, such as ³H, ¹⁴C, ³²p, ³⁵S, or ¹²⁵I, a fluorescent or chemiluminescent compound, such as fluorescein isothiocyanate, rhodamine, or luciferin; biotin (which enables detection of the antibody with an agent that binds to biotin, such as avidin; or an enzyme (either by chemical coupling or polypeptide fusion), such as alkaline phosphatase, beta-galactosidase or horseradish peroxidase.

Any method known in the art for separately conjugating the antibody to the detectable moiety may be employed, including those methods described by Hunter et al. (1962); David et al. (1974); Pain et al. (1981); and Nygren (1982).

The antibodies of the present invention may be employed in any known assay method, such as competitive binding assays, direct and indirect sandwich assays, and immunoprecipitation assays (Zola, 1987). For instance the antibodies may be used in the diagnostic assays described herein.

Additionally, antibodies may be used in competitive binding assays. These assays rely on the ability of a labeled standard (which may be a purified target antigen or an immunologically reactive portion thereof) to compete with the test sample analyte for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex (see for example U.S. Pat. No. 4,376,110). The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

Some specific antibodies that maybe used in conjunction with methods of the current invention include but are not limited to those listed in Table 1 of U.S. Publication 2006/0040338 or Table 1 of Mandell (2003), each incorporated herein by reference. For example, antibodies for use in the invention may include or may exclude 1, 2, 3, 4, 5, 6, 7, 8, 9, 10 or more of Akt (pS472/pS473) Phospho-Specific (PKBa) Antibodies, Caveolin (pY14) Phospho-Specific Antibodies, Cdk1/Cdc2 (pY15) Phospho-Specific Antibodies, eNOS (PS1177) Phospho-Specific Antibodies, eNOS (pT495) Phospho-Specific Antibodies, ERK1/2 (pT202/pY204) Phospho-Specific Antibodies, (p44/42 MAPK) FAK (pY397) Phospho-Specific Antibodies, IkBa (pS32/pS36) Phospho-Specific Antibodies, Integrin b3 (pY759) Phospho-Specific Antibodies, JNK (pT183/pY185) Phospho-Specific Antibodies, Lck (pY505) Phospho-Specific Antibodies, p38 MAPK (pT180/pY182) Phospho-Specific Antibodies, p120 Catenin (pY228) Phospho-Specific Antibodies, p120 Catenin (pY280) Phospho-Specific Antibodies, p120 Catenin (pY96) Phospho-Specific Antibodies, Paxillin (pY118) Phospho-Specific Antibodies, Phospholipase Cg (pY783) Phospho-Specific Antibodies, PKARIIb (pS114) Phospho-Specific Antibodies, 14-3-3 Binding Motif Phospho-specific Antibodies, 4E-BP1 Phospho-specific Antibodies, AcCoA Carboxylase (Acetyl CoA) Phospho-specific Antibodies, Adducin Phospho-specific Antibodies, AFX Phospho-specific Antibodies, AIK (Aurora 2) Phospho-specific Antibodies, Akt (PKB) Phospho-specific Antibodies, Akt (PKB) Substrate Phospho-specific Antibodies, ALK Phospho-specific Antibodies, AMPK alpha Phospho-specific Antibodies, AMPK beta1 Phospho-specific Antibodies, APP Phospho-specific Antibodies, Arg-X-Tyr/Phe-X-pSer Motif Phospho-specific Antibodies, Arrestin 1 beta Phospho-specific Antibodies, ASK1 Phospho-specific Antibodies, ATF-2 Phospho-specific Antibodies, ATM/ATR Substrate Phospho-specific Antibodies, Aurora 2 (AIK) Phospho-specific Antibodies, Bad Phospho-specific Antibodies, Bcl-2 Phospho-specific Antibodies, Bcr Phospho-specific Antibodies, Bim EL Phospho-specific Antibodies, BLNK Phospho-specific Antibodies, BMK1 (ERK5) Phospho-specific Antibodies, BRCA1 Phospho-specific Antibodies, Btk Phospho-specific Antibodies, C/EBP alpha Phospho-specific Antibodies, C/EBP beta Phospho-specific Antibodies, c-Ab1 Phospho-specific Antibodies, CAKb Phospho-specific Antibodies, Caldesmon Phospho-specific Antibodies, CaM Kinase II Phospho-specific Antibodies, Cas p130 Phospho-specific Antibodies, Catenin beta Phospho-specific Antibodies, Catenin p120 Phospho-specific Antibodies, Caveolin 1 Phospho-specific Antibodies, Caveolin 2 Phospho-specific Antibodies, Caveolin Phospho-specific Antibodies, c-Cb1 Phospho-specific Antibodies, CD117 (c-Kit) Phospho-specific Antibodies, CD19 Phospho-specific Antibodies, cdc2 p34 Phospho-specific Antibodies, cdc2 Phospho-specific Antibodies, cdc25 C Phospho-specific Antibodies, cdk1 Phospho-specific Antibodies, cdk2 Phospho-specific Antibodies, CDKs Substrate Phospho-specific Antibodies, CENP-A Phospho-specific Antibodies, c-erbB-2 Phospho-specific Antibodies, Chk1 Phospho-specific Antibodies, Chk2 Phospho-specific Antibodies, c-Jun Phospho-specific Antibodies, c-Kit (CD117) Phospho-specific Antibodies, c-Met Phospho-specific Antibodies, c-Myc Phospho-specific Antibodies, Cofilin 2 Phospho-specific Antibodies, Cofilin Phospho-specific Antibodies, Connexin 43 Phospho-specific Antibodies, Cortactin Phospho-specific Antibodies, CPI-17 Phospho-specific Antibodies, cPLA2 Phospho-specific Antibodies, c-Raf (Raf1) Phospho-specific Antibodies, CREB Phospho-specific Antibodies, c-Ret Phospho-specific Antibodies, CrkII Phospho-specific Antibodies, CrkL Phospho-specific Antibodies, Cyclin Bi Phospho-specific Antibodies, DARPP-32 Phospho-specific Antibodies, DNA-topoisomerase II alpha Phospho-specific Antibodies, Dok-2 p56 Phospho-specific Antibodies, eEF2 Phospho-specific Antibodies, eEF2k Phospho-specific Antibodies, EGF Receptor (EGFR) Phospho-specific Antibodies, eIF2 alpha Phospho-specific Antibodies, eIF2B epsilon Phospho-specific Antibodies, eIF4 epsilon Phospho-specific Antibodies, eIF4 gamma Phospho-specific Antibodies, Elk-1 Phospho-specific Antibodies, eNOS Phospho-specific Antibodies, EphA3 Phospho-specific Antibodies, Ephrin B Phospho-specific Antibodies, erbB-2 Phospho-specific Antibodies, ERK1/ERK2 Phospho-specific Antibodies, ERK5 (BMK1) Phospho-specific Antibodies, Estrogen Receptor alpha (ER-a) Phospho-specific Antibodies, Etk Phospho-specific Antibodies, Ezrin Phospho-specific Antibodies, FADD Phospho-specific Antibodies, FAK Phospho-specific Antibodies, FAK2 Phospho-specific Antibodies, Fc gamma RIIb Phospho-specific Antibodies, FGF Receptor (FGFR) Phospho-specific Antibodies, FKHR Phospho-specific Antibodies, FKHRL1 Phospho-specific Antibodies, FLT3 Phospho-specific Antibodies, FRS2-alpha Phospho-specific Antibodies, Gab1 Phospho-specific Antibodies, Gab2 Phospho-specific Antibodies, GABA B Receptor Phospho-specific Antibodies, GAP-43 Phospho-specific Antibodies, GATA4 Phospho-specific Antibodies, GFAP Phospho-specific Antibodies, Glucocorticoid Receptor Phospho-specific Antibodies, GluR1 (Glutamate Receptor 1) Phospho-specific Antibodies, GluR2 (Glutamate Receptor 2) Phospho-specific Antibodies, Glycogen Synthase Phospho-specific Antibodies, GRB10 Phospho-specific Antibodies, GRK2 Phospho-specific Antibodies, GSK-3 alpha/beta Phospho-specific Antibodies, GSK-3 alpha Phospho-specific Antibodies, GSK-3 beta (Glycogen Synthase Kinase) Phospho-specific Antibodies, GSK-3 beta Phospho-specific Antibodies, GSK-3 Phospho-specific Antibodies, H2A.X Phospho-specific Antibodies, Hck Phospho-specific Antibodies, HER-2 (ErbB2) Phospho-specific Antibodies, Histone H1 Phospho-specific Antibodies, Histone H2A.X Phospho-specific Antibodies, Histone H2B Phospho-specific Antibodies, Histone H3 Phospho-specific Antibodies, HMGN1 (HMG-14) Phospho-specific Antibodies, Hsp27 (Heat Shock Protein 27) Phospho-specific Antibodies, IkBa (I kappa B-alpha) Phospho-specific Antibodies, Integrin alpha-4 Phospho-specific Antibodies, Integrin beta-1 Phospho-specific Antibodies, Integrin beta-3 Phospho-specific Antibodies, IR (Insulin Receptor) Phospho-specific Antibodies, IR/IGF1R (Insulin/Insulin-Like Growth Factor-1 Receptor) Phospho-specific Antibodies, IRS-1 Phospho-specific Antibodies, IRS-2 Phospho-specific Antibodies, Jak1 Phospho-specific Antibodies, Jak2 Phospho-specific Antibodies, JNK (SAPK) Phospho-specific Antibodies, Jun Phospho-specific Antibodies, KDR Phospho-specific Antibodies, Keratin 18 Phospho-specific Antibodies, Keratin 8 Phospho-specific Antibodies, Kinase Substrate Phospho-specific Antibodies, Kip1 p27 Phospho-specific Antibodies, LAT Phospho-specific Antibodies, Lck Phospho-specific Antibodies, Leptin Receptor Phospho-specific Antibodies, LKB1 Phospho-specific Antibodies, Lyn Phospho-specific Antibodies, MAP Kinase/CDK Substrate Phospho-specific Antibodies, MAP Kinase p38 Phospho-specific Antibodies, MAP Kinase p44/42 Phospho-specific Antibodies, MAPKAP Kinase 1a (Rsk1) Phospho-specific Antibodies, MAPKAP Kinase 2 Phospho-specific Antibodies, MARCKS Phospho-specific Antibodies, Maturation Promoting Factor (MPF) Phospho-specific Antibodies, M-CSF Receptor Phospho-specific Antibodies, MDM2 Phospho-specific Antibodies, MEK1/MEK2 Phospho-specific Antibodies, MEK1 Phospho-specific Antibodies, MEK2 Phospho-specific Antibodies, MEK4 Phospho-specific Antibodies, MEK7 Phospho-specific Antibodies, Met Phospho-specific Antibodies, MKK3/MKK6 Phospho-specific Antibodies, MKK4 (SEK1) Phospho-specific Antibodies, MKK7 Phospho-specific Antibodies, MLC Phospho-specific Antibodies, MLK3 Phospho-specific Antibodies, Mnk1 Phospho-specific Antibodies, MPM2 Phospho-specific Antibodies, MSK1 Phospho-specific Antibodies, mTOR Phospho-specific Antibodies, Myelin Basic Protein (MBP) Phospho-specific Antibodies, Myosin Light Chain 2 Phospho-specific Antibodies, MYPT1 Phospho-specific Antibodies, neu (Her2) Phospho-specific Antibodies, Neurofilament Phospho-specific Antibodies, NFAT1 Phospho-specific Antibodies, NF-kappa B p65 Phospho-specific Antibodies, Nibrin (p95/NBS1) Phospho-specific Antibodies, Nitric Oxide Synthase Endothelial (eNOS) Phospho-specific Antibodies, Nitric Oxide Synthase Neuronal (nNOS) Phospho-specific Antibodies, NMDA Receptor 1 (NMDAR1) Phospho-specific Antibodies, NMDA Receptor 2B (NMDA NR2B) Phospho-specific Antibodies, nNOS Phospho-specific Antibodies, NPM Phospho-specific Antibodies, Opioid Receptor delta Phospho-specific Antibodies, Opioid Receptor mu Phospho-specific Antibodies, p53 Phospho-specific Antibodies, PAK1/2/3 Phospho-specific Antibodies, PAK2 Phospho-specific Antibodies, Paxilin Phospho-specific Antibodies, Paxillin Phospho-specific Antibodies, PDGF Receptor alpha/beta Phospho-specific Antibodies, PDGF Receptor alpha Phospho-specific Antibodies, PDGF Receptor beta Phospho-specific Antibodies, PDGFRb (Platelet Derived Growth Factor Receptor beta) Phospho-specific Antibodies, PDK1 Docking Motif Phospho-specific Antibodies, PDK1 Phospho-specific Antibodies, PDK1 Substrate Phospho-specific Antibodies, PERK Phospho-specific Antibodies, PFK-2 Phospho-specific Antibodies, Phe Phospho-specific Antibodies, Phospholamban Phospho-specific Antibodies, Phospholipase C gamma-1 Phospho-specific Antibodies, Phosphotyrosine IgG Phospho-specific Antibodies, phox p40 Phospho-specific Antibodies, PI3K Binding Motif p85 Phospho-specific Antibodies, Pin1 Phospho-specific Antibodies, PKA Substrate Phospho-specific Antibodies, PKB (Akt) Phospho-specific Antibodies, PKB (Akt) Substrate Phospho-specific Antibodies, PKC alpha/beta II Phospho-specific Antibodies, PKC alpha Phospho-specific Antibodies, PKC delta/theta Phospho-specific Antibodies, PKC delta Phospho-specific Antibodies, PKC epsilon Phospho-specific Antibodies, PKC eta Phospho-specific Antibodies, PKC gamma Phospho-specific Antibodies, PKC Phospho-specific Antibodies, PKC Substrate Phospho-specific Antibodies, PKC theta Phospho-specific Antibodies, PKC zeta/lambda Phospho-specific Antibodies, PKD (PKC mu) Phospho-specific Antibodies, PKD2 Phospho-specific Antibodies, PKR Phospho-specific Antibodies, PLC beta 3 Phospho-specific Antibodies, PLC gamma 1 Phospho-specific Antibodies, PLC gamma 2 Phospho-specific Antibodies, PLD1 Phospho-specific Antibodies, PP1 alpha Phospho-specific Antibodies, PP2A Phospho-specific Antibodies, PPAR Alpha Phospho-specific Antibodies, PRAS40 Phospho-specific Antibodies, Presenilin-2 Phospho-specific Antibodies, PRK2 (pan-PDK1 phosphorylation site) Phospho-specific Antibodies, Progesterone Receptor Phospho-specific Antibodies, Protein Kinase A RII (PKARII) Phospho-specific Antibodies, Protein Kinase B Phospho-specific Antibodies, Protein Kinase B Substrate Phospho-specific Antibodies, Protein Kinase C alpha (PKCa) Phospho-specific Antibodies, Protein Kinase C epsilon (PKCe) Phospho-specific Antibodies, PTEN Phospho-specific Antibodies, Pyk2 Phospho-specific Antibodies, Rac1/cdc42 Phospho-specific Antibodies, Rac-Pk Phospho-specific Antibodies, Rac-Pk Substrate Phospho-specific Antibodies, Rad 17 Phospho-specific Antibodies, Rad17 Phospho-specific Antibodies, Raf-1 Phospho-specific Antibodies, Ras-GRF1 Phospho-specific Antibodies, Rb (Retinoblastoma Protein) Phospho-specific Antibodies, Ret Phospho-specific Antibodies, Ribosomal Protein S6 Phospho-specific Antibodies, RNA polymerase II Phospho-specific Antibodies, Rsk p90 Phospho-specific Antibodies, Rsk1 (MAPKAP K1a) Phospho-specific Antibodies, Rsk3 Phospho-specific Antibodies, S6 Kinase Phospho-specific Antibodies, S6 Kinase p70 Phospho-specific Antibodies, S6 peptide Substrate Phospho-specific Antibodies, SAPK (JNK) Phospho-specific Antibodies, SAPK2 (Stress-activated Protein Kinase SKK3 MKK3) Phospho-specific Antibodies, SEK1 (MKK4) Phospho-specific Antibodies, Serotonin N-AT Phospho-specific Antibodies, Serotonin-N-AT Phospho-specific Antibodies, SGK Phospho-specific Antibodies, Shc Phospho-specific Antibodies, SHIP1 Phospho-specific Antibodies, SHP-2 Phospho-specific Antibodies, SLP-76 Phospho-specific Antibodies, Smad1 Phospho-specific Antibodies, Smad2 Phospho-specific Antibodies, SMC1 Phospho-specific Antibodies, SMC3 Phospho-specific Antibodies, SOX-9 Phospho-specific Antibodies, Src Family Negative Regulatory Site Phospho-specific Antibodies, Src Family Phospho-specific Antibodies, Src Phospho-specific Antibodies, Stat1 Phospho-specific Antibodies, Stat2 Phospho-specific Antibodies, Stat3 Phospho-specific Antibodies, Stat4 Phospho-specific Antibodies, Stat5 Phospho-specific Antibodies, Stat5A/Stat5B Phospho-specific Antibodies, Stat5ab Phospho-specific Antibodies, Stat6 Phospho-specific Antibodies, Syk Phospho-specific Antibodies, Synapsin Phospho-specific Antibodies, Synapsin site 1 Phospho-specific Antibodies, Tau Phospho-specific Antibodies, Tie 2 Phospho-specific Antibodies, Trk A Phospho-specific Antibodies, Troponin I Cardiac Phospho-specific Antibodies, Tuberin Phospho-specific Antibodies, Tyk 2 Phospho-specific Antibodies, Tyrosine Hydroxylase Phospho-specific Antibodies, Tyrosine Phospho-specific Antibodies, VASP Phospho-specific Antibodies, Vav1 Phospho-specific Antibodies, Vav3 Phospho-specific Antibodies, VEGF Receptor 2 Phospho-specific Antibodies, or Zap-70 Phospho-specific Antibodies, as well as non-phosphorylation specific counterparts.

### C. Humanized Antibodies

As discussed previously, antibodies for use in the methods of the invention may be polyclonal or monoclonal antibodies or fragments thereof. However, for certain therapeutic purposes aspects the antibodies are humanized such that they do not elicit an immune response in a subject being treated. Such humanized antibodies may also be used according to the current invention and methods for generating such antibodies are well known to those of skill in the art (Jones et al., 1986); Riechmann et al., 1988; Verhoeyen et al., 1988).

### D. Single Chain Antibodies

Single chain antibodies (SCAs) are genetically engineered proteins designed to expand on the therapeutic and diagnostic applications possible with monoclonal antibodies. SCAs have the binding specificity and affinity of monoclonal antibodies and, in their native form, are about one-fifth to one-sixth of the size of a monoclonal antibody, typically giving them very short half-lives. SCAs offer some benefits compared to most monoclonal antibodies, including their ability to be directly fused with a polypeptide that may be used for detection (e.g., luciferase or fluorescent proteins). In addition to these benefits, fully-human SCAs can be isolated directly from human SCA libraries without the need for costly and time consuming “humanization” procedures.

Single-chain recombinant antibodies (scFvs) consist of the antibody VL and VH domains linked by a designed flexible peptide tether (Atwell et al., 1999). Compared to intact IgGs, scFvs have the advantages of smaller size and structural simplicity with comparable antigen-binding affinities, and they can be more stable than the analogous 2-chain Fab fragments (Colcher et al., 1998; Adams and Schier, 1999).

The variable regions from the heavy and light chains (VH and VL) are both approximately 110 amino acids long. They can be linked by a 15 amino acid linker or longer with the sequence, for example, which has sufficient flexibility to allow the two domains to assemble a functional antigen binding pocket. In specific embodiments, addition of various signal sequences allows the scFv to be targeted to different organelles within the cell, or to be secreted. Addition of the light chain constant region (Ck) allows dimerization via disulfide bonds, giving increased stability and avidity. Thus, for a single chain Fv (scFv) SCA, although the two domains of the Fv fragment are coded for by separate genes, it has been proven possible to make a synthetic linker that enables them to be made as a single protein chain scFv (Bird et al., 1988; Huston et al., 1988) by recombinant methods. Furthermore, they are frequently used due to their ease of isolation from phage display libraries and their ability to recognize conserved antigens (for review, see Adams and Schier, 1999). Thus, in some aspects of the invention, an antibody may be an SCA that is isolated from a phage display library rather that generated by the more traditional antibody production techniques described above.

### IV. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### RPPA Methods And Analyses

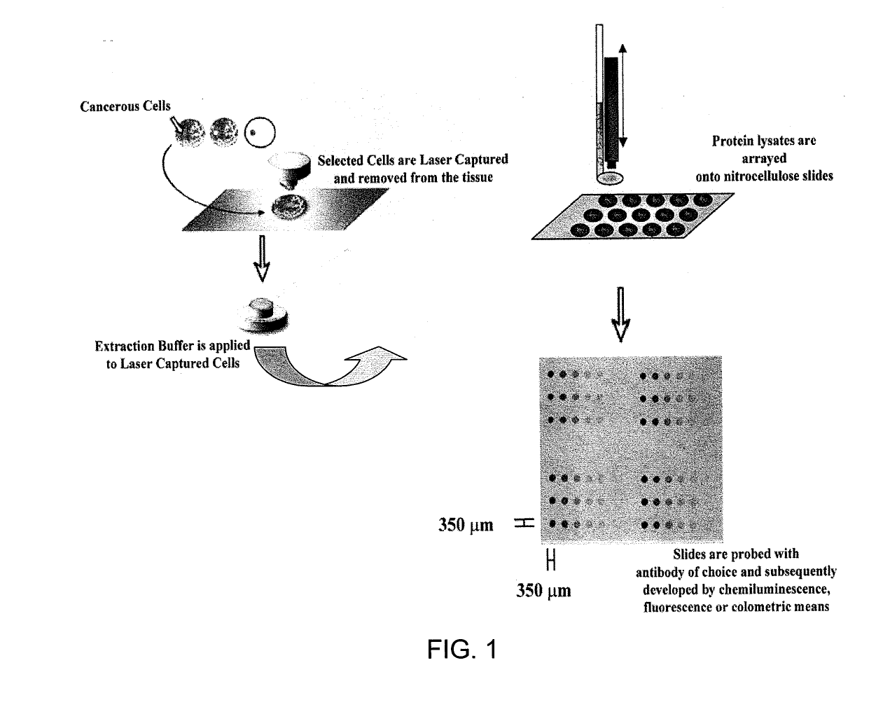

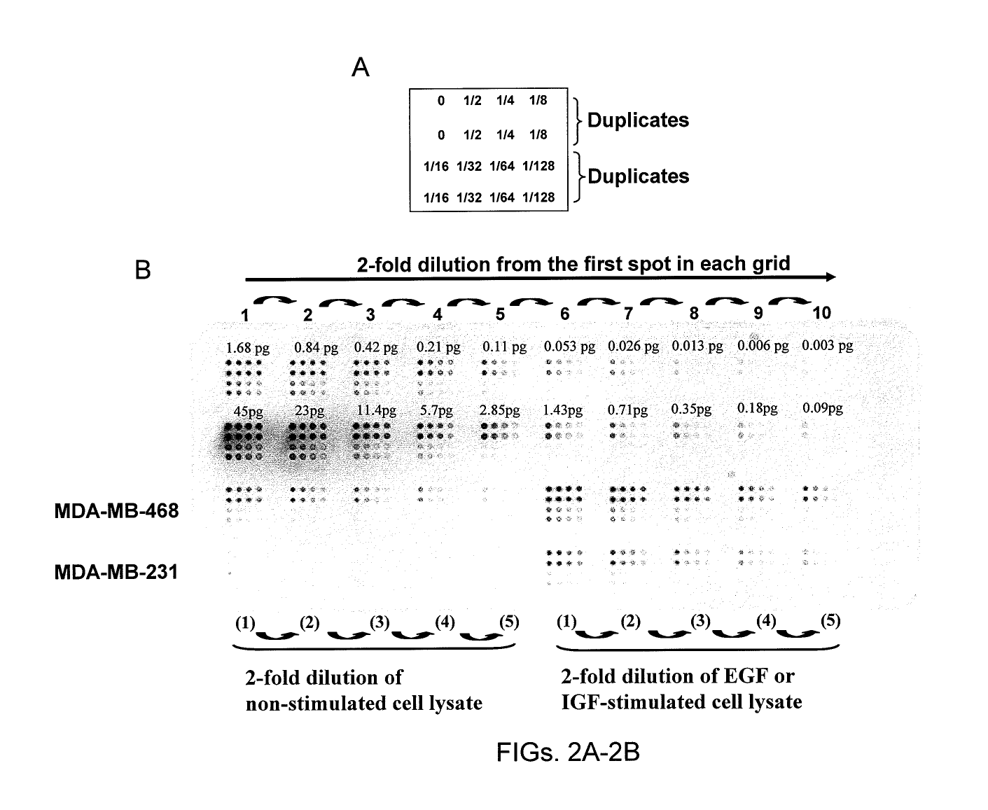

RPPA Method: General methods for RPPA are exemplified in FIG. 1. Protein lysates will be obtained by mixing tissue sample material with 1 ml of lysis buffer/40 milligrams of frozen tissue and then serially diluted (8 serial dilutions: full strength, ½, ¼, ⅛, 1/16, 1/32, 1/64, 1/128) with additional lysis buffer. Dilutions will be made with Tecan liquid handling robot. This material is printed onto nitrocellulose-coated glass slides (FAST Slides, Schleicher & Schuell BioScience, Inc. USA, Keene, N.H.) with an automated GeneTac arrayer (Genomic Solutions, Inc., Ann Arbor, Mich.) that transfers 1 nl of protein lysate per touch. As many as 80 samples can be spotted in 8 serial dilutions on a single slide. The serial dilutions provide a slope and intercept allowing relative quantification of individual proteins. This is compared to control peptides (in house) allowing absolute quantification (see FIGS. 2A-2B). After slide printing, the same stringent conditions for slide blocking, blotting and antibody incubation used for western blotting are applied prior to the addition of the primary antibody. The DAKO (Copenhagen, Denmark) signal amplification system can be used to detect and amplify antibody-binding intensity.

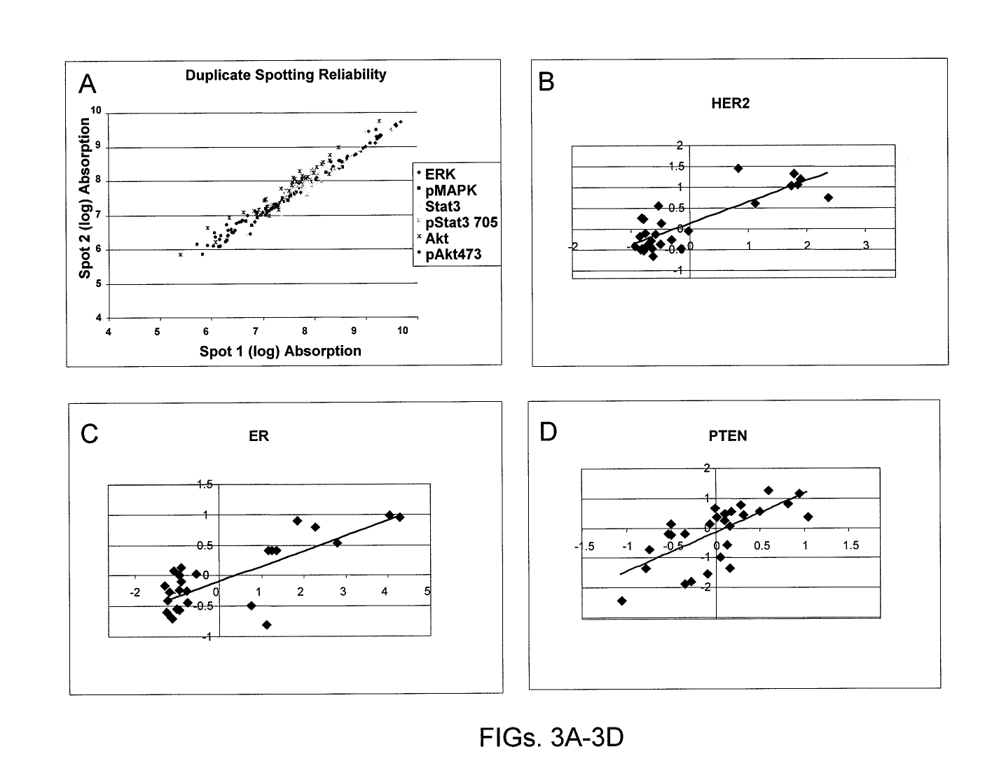

Signal intensity is measured by scanning the slides and quantifying with the MicroVigene automated RPPA software (VigeneTech Inc., Massachusetts) to generate sigmoidal signal intensity-concentration curves for each sample. To accurately determine absolute protein concentrations, standard signal intensity-concentration curves for purified proteins/recombinant peptides of known concentration are generated for comparison with the samples in which protein concentrations are unknown. It is demonstrated that RPPAs are quantitative, sensitive, and reproducible. FIG. 3A illustrates the reproducibility of RPPA and FIGS. 3B-3D demonstrate that measurements with RPPA correlates with previously available assay methods. RPPA may also be validated with mTOR, erk, p38, GSK3 and JNK as stable loading controls.

Quantified protein expression data is analyzed, using programs and algorithms identical to those used for analysis of gene expression arrays. The data is analyzed for the presence of clusters based on differential protein expression using methods available in the R statistical software package (cran.r-project.org). A variety of clustering methods (including hierarchical clustering, K-means, independent component analysis, mutual information, and gene shaving) are used to classify samples into statistically similar groups. For example, Xcluster (SMD software, Paulo Alto, Calif.) and TreeView (University of Glasgow, Glasgow, Scotland) software may be used to put all this data together into unsupervised hierarchical clusters or heat maps which arrange the samples in terms of similarity in protein expression and activation. Robustness and statistical significance of these groups may be evaluated by bootstrap data resampling (Kerr and Churchill, 2001). In addition to primary clustering analysis based on all proteins, secondary bootstrap-resampled clustering analyses may be performed using proteins in a signaling pathway of interest.

In order for a cluster that is statistically significant based on bootstrap resampling to represent an important subtype of breast cancer, the cluster should contain samples from at least 5 patients. For instance, using the 80 samples, as in Example 4, a breast cancer subtype with 10% prevalence will have a 90% probability of contributing at least 5 samples to the study population. Thus, the proposed patient sample should be sufficient to detect subtypes with at least 10% prevalence. A potential problem is batch effect since analyses are performed on more slides than can be printed at one time. However, evidence suggests that inter-slide variation is minimal (R2>0.8) when slides are printed at different times and stained with the same antibody. An advantage of RPPA is that as new, potentially relevant proteins are identified stored sample preparations/plates may be used to probe for these novel proteins and the data can be incorporated into the dataset for analysis. Thus, the sample set will be continuously enriched. As only a small amount of lysate is required, the samples can accommodate analysis of up to a thousand antibodies easily.

Patient samples are typically linked to an oncology database such as the Breast Medical Oncology Database, which includes patient characteristics and outcome information (response to PC, type of therapy, etc.). These data can be correlated with the RPPA clusters using standard statistical methods, including Fisher's exact test, analysis of variance, and Cox proportional hazards models for time to recurrence. In this way, it can be determined if clusters of patient samples generated by RPPAs have clinical significance and correlate with a specific endpoint: e.g., pathological complete response (pCR). Supervised statistical approaches may also be employed to assist in building the pCR predictor. Adequate power to determine differences will require a ‘training set’ (e.g., 80 samples). In addition, the inventors contemplate identifying kinase signaling patterns in chemotherapy-unresponsive tumors that can be targeted to augment the efficacy of cytotoxic treatment.

### Example 2

### Predictive Markers For Breast Cancer Prognosis

An algorithm is developed to predict clinical outcome in patients with hormone receptor positive breast cancer. The algorithm is developed and validated in a set of breast tumors and uses 5 protein markers: estrogen receptor (ER), progesterone receptor (PR), and phosphorylation of Akt, p38, and mammalian target of rapamycin (mTor). ER is currently assayed as a dichotomous variable and the validity of this approach is being questioned at present by, for example, the Food and Drug Administration. Lysate arrays treat ER as a continuous variable and data suggests that the quantity of ER protein is a major driver of outcome after anti-hormonal therapy for hormone receptor-positive breast cancer. Thus, ER quantification using lysate array technology may be capable of improving upon the current immunohistochemical assays for determining the hormone responsiveness of breast tumors.

Reverse phase tissue lysate arrays and Microvigene software™ are used to quantify the expression of estrogen receptor alpha (ER) and 36 total/activated components of the HER2, phosphatidylinositol-3-kinase (P13K), mitogen-activated protein kinase (MAPK), and STAT pathways in 64 hormone receptor-positive breast cancers and 40 breast cancer cell lines. Clustering is performed with Xcluster™ and Treeview™. Forty seven of the 64 hormone receptor-positive breast cancer patients are treated with adjuvant hormone therapy and 43 with chemotherapy. There are 12 recurrences including 5 patients diagnosed with metastases within 0-3 months of diagnosis. Unsupervised analysis using the expression of all 37 proteins reveal two large subclusters of hormone receptor-positive breast cancers. One large cluster is composed of tumors with lower ER expression levels and was driven by an antibody group composed mostly of phosphoproteins indicative of activated growth factor signaling pathways. Thus, there are significant inverse correlations between ER expression and the expression and activation of components of the PI3K/MAPK pathways including EGFR, src, AKT, 4EBP1, and PKC alpha (p under 0.05 for each). Similar inverse correlations were seen in 40 assayed breast cancer cell lines. The clinicoproteomic predictors of relapse among hormone receptor-positive breast cancers are nuclear grade (p=0.001), low expression of ER (p=0.04), low p38 phosphorylation (p=0.02), and high p53 (p=0.02). There also is a trend (p under 0.1) to the association of low MAPK and S6 phosphorylation, low p27, and high cyclin B1 with relapse. Using quantification with these 7 antibodies to perform a supervised analysis a small group of p53-high, cyclin B1-high, ER-low hormone receptor-positive breast cancers with a 75% likelihood of relapse are identified, significantly greater than in other tumors (p<0.003). Since 10 of 12 relapses occur in 26 grade 3 hormone receptor-positive breast tumors, a ‘grade 3’ protein signature associated with a recurrence-free survival at 20 months of 17% compared to 100% in other patients (p=0.002).

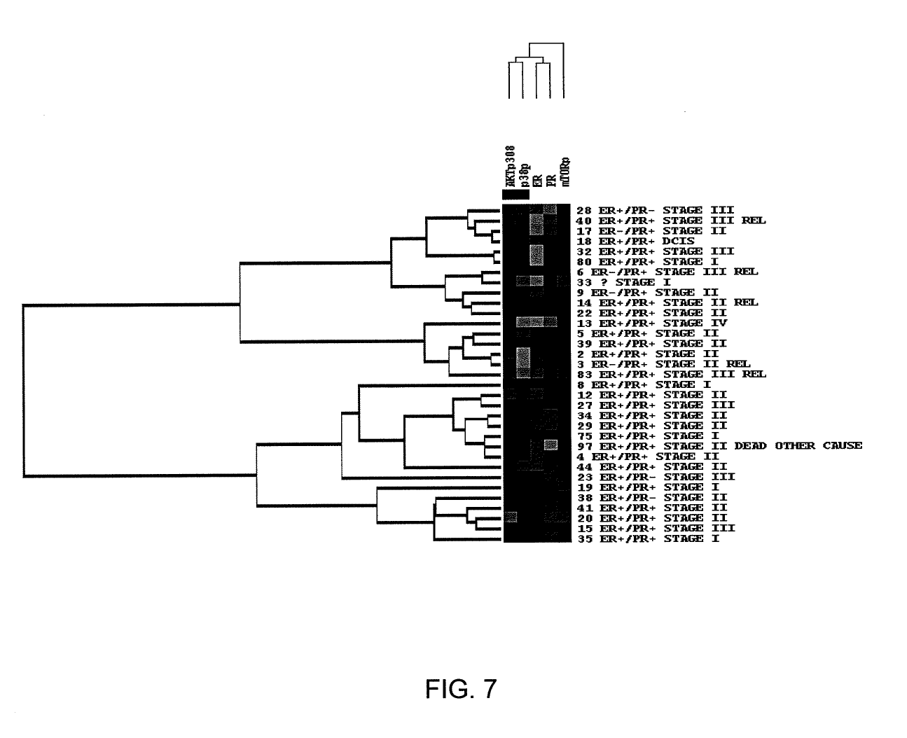

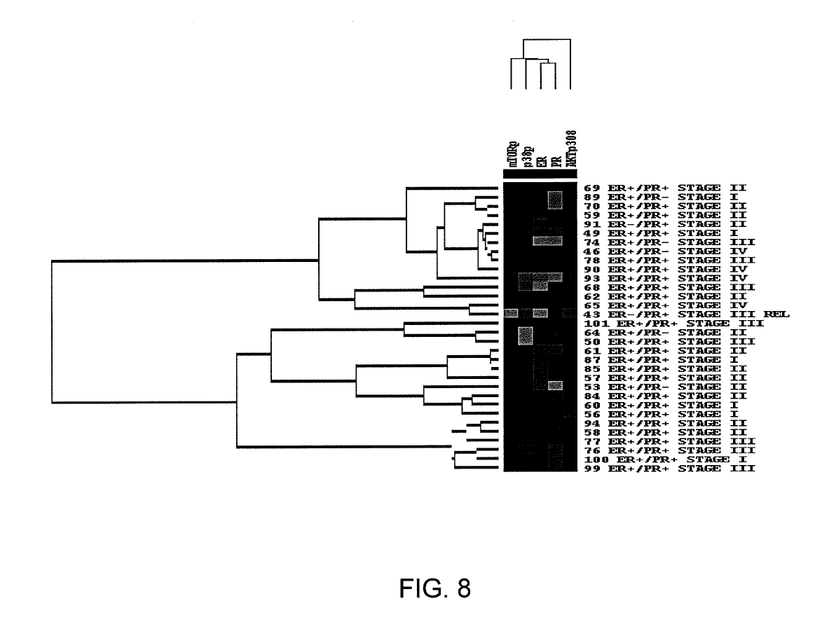

As described above, an algorithm to predict outcome in all patients with hormone receptor positive breast cancers is developed. The algorithm comprises 5 protein markers: estrogen receptor (ER), progesterone receptor (PR), and phosphorylation of Akt, p38, and mammalian target of rapamycin (mTor) (FIGS. 7 and 8). In addition, lysate arrays treat ER as a continuous variable and studies suggest that the quantity of ER protein is the major driver of outcome after antihormonal therapy for hormone receptor-positive breast cancer. Thus, ER quantification using lysate array technology may be capable of improving upon the current immunohistochemical standard approach of determining the hormone responsiveness of breast tumors. Patients may stratify as follows:

1. Patients with high ER and low PI3K may be extremely sensitive to only tamoxifen or aromatase inhibitors.

2. Patients with low ER and high PI3K may be sensitive to PI3K inhibitors combined with aromatase inhibitors.

3. Patients with low ER and high PI3K may need hormonal manipulation and chemotherapy.

4. Patients with low ER and high PI3K might be sensitive to agents that decrease ER levels (these are in clinical use) rather than aromatase inhibitors.

Further, as described in the previous sections, the tissue lysate array-based approach has clinical application in stratifying patients with hormone receptor positive breast cancer to a treatment decision based on quantification of ER and activation status of various components of kinase signaling pathways.

### Example 3

### Predictive Markers For Ovarian Cancer Prognosis

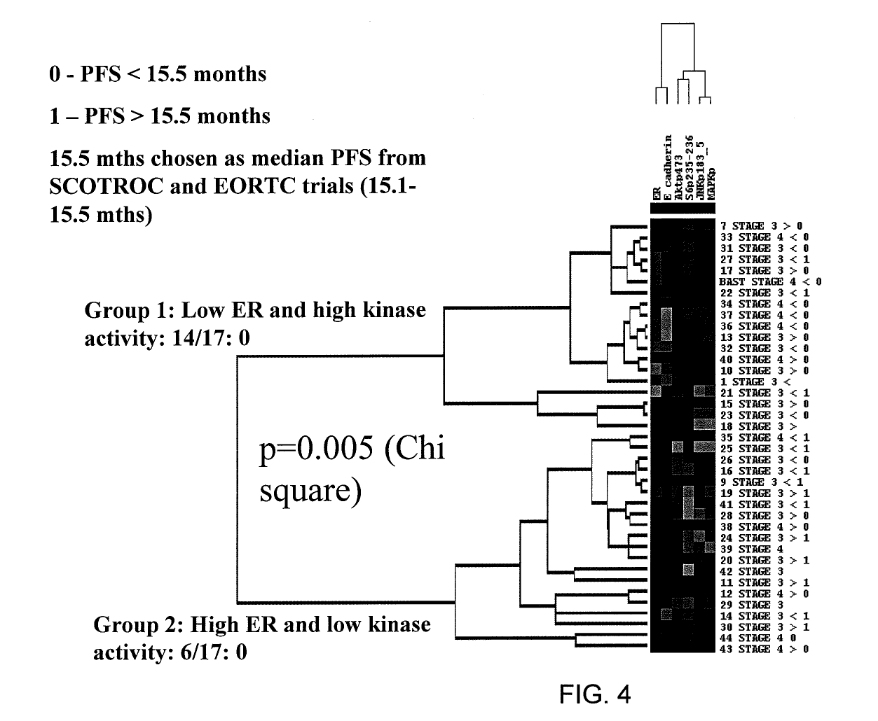

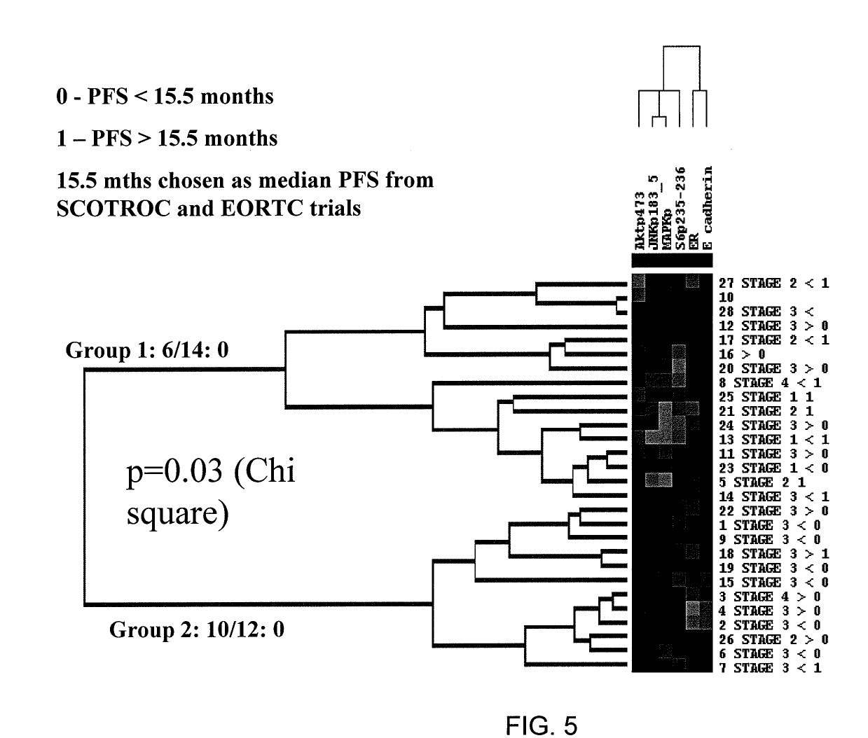

Ovarian cancer prognostic and predictive signatures are developed using reverse phase tissue lysate array-based quantification of the expression and activation of protein members of kinase signaling pathways (e.g., phosphatidylinositol-3-kinase (PI3K)/Akt and mitogen activated protein kinase (MAPK)) and steroid signaling pathways. Signatures may be useful as a guide to patient prognosis and also for prediction of the likelihood that individuals with ovarian cancer will derive benefit from specific chemotherapies and potentially targeted therapies. Reverse phase tissue lysate arrays and Microvigene software™ are used to quantify the expression of estrogen receptor alpha (ER), progesterone receptor (PR), and 36 total/activated components of the HER2, phosphatidylinositol-3-kinase (PI3K), mitogen-activated protein kinase (MAPK), and STAT pathways in a test set of 44 human ovarian cancers (FIG. 4) and a validation set of 28 human ovarian cancers (FIG. 5). The majority are stage III/IV high-grade cancers in patients treated with surgery followed by platinum-based chemotherapy. Clustering, both supervised and unsupervised, is performed with Xcluster™ and Treeview™. A supervised algorithm to predict outcome in high grade human ovarian cancer after surgery and platinum-based chemotherapy is developed and validated in the preliminary validation set. The algorithm comprises 6 protein markers: estrogen receptor (ER), E cadherin, and phosphorylation of Akt (serine 473), MAPK (44/42), c-jun N-terminal kinase (JNK), and S6. This signature is prognostic after surgery and chemotherapy for high-grade ovarian cancer patients.

### Example 4

### RPPA Signaling Signatures From Frozen Breast Cancer Tissue Samples

A protein signaling signature will be characterized in multiple frozen breast cancer samples by unsupervised hierarchical clustering of reverse phase protein arrays (RPPAs). Lysates of several frozen breast cancer fine needle aspirate (FNA) samples are arrayed on slides followed by probing with validated monospecific antibodies to multiple proteins and subsequent signal detection and quantification using Microvigene software (VigeneTech Inc., Massachusetts), we can use Xcluster (SMD software, Paulo Alto, Calif.) and TreeView (University of Glasgow, Glasgow, Scotland) software to put all this data together into unsupervised hierarchical clusters or heat maps which arrange the samples in terms of similarity in protein expression and activation. Using this approach, there is evidence of a correlation with patient outcome.

To classify breast cancer by characterizing the functional proteomic expression/activation signature of 3 signal transduction cascades (PI13K, JAK/STAT, and MAPK), the hormone receptors ER and PR, and the proteins GST, TOPO, survivin, and tau. Signaling through the PI3K, JAK/STAT, and MAPK signaling pathways, and the proteins ER, PR, GST, TOPO, survivin, and tau all have an important role in breast cancer. The simultaneous characterization of this ‘proteome’ using RPPA in patient samples will allow us to cluster breast cancers into distinct molecular types and identify proteins which together play an important role in the cancer phenotype.

Tissue Collection: 80 snap frozen breast cancer FNAs collected from the primary tumor prior to preopertive chemotherapy (PC) on IRB-approved protocol LAB 99-402 will be studied by RPPAs using 48 antibodies. These antibodies provide quantitative analysis of the signaling pathways noted above in detail as well additional signaling events implicated in breast and other cancers.

Information obtained from pathologic surgical specimens from a completely independent group of 50 patients treated with PC in whom pre-PC biopsies are obtained on LAB 99-402 and correlate the tumor response to PC with the PC response predictor constructed above from the functional proteomic expression/activation signature of 3 signal transduction cascades (PI3K, JAK/STAT, and MAPK), the hormone receptors ER and PR, and the proteins GST, TOPO, survivin, and tau in these tumors.

### Example 5

### RPPA Functional Proteomic Patterns To Prediction Clinical Behavior of Breast Cancer And Ovarian Cancer

The inventors have utilized RPPA to study functional proteomic patterns of relevance to prediction of the clinical behavior of breast cancer and ovarian cancer using antibodies of Table 1 that have been validated or are in the process of being validated for use in RPPA. These antibodies detect proteins that belong to the groups above and were selected to develop a coordinate picture of expression and activation (e.g., phosphorylation (p)) of signaling processes that play an important role in breast and ovarian carcinogenesis. The inventors have analyzed protein lysates from:

1. 116 early stage hormone receptor-positive breast cancers, treated with adjuvant hormonal therapy (65 patients) vs. untreated (51 patients).

2. 43 early stage HER2 amplified breast cancers, treated with adjuvant cytotoxic chemotherapy.

3. 52 early stage triple receptor-negative breast cancers, treated with adjuvant cytotoxic chemotherapy.

4. 112 high grade ovarian cancers obtained at primary surgery in patients with newly diagnosed EOC who were then treated in a standard fashion with carboplatin and paclitaxel.

Supervised analysis of the RPPA data was performed using standard and novel statistical approaches.

In ovarian cancer, such analysis approaches have identified two overlapping groups of functional proteomic biomarkers with excellent sensitivity, specificity, positive, and negative predictive values for prediction of poor patient prognosis as a result of primary ovarian cancer ‘platinum resistance,’ i.e., disease progression within six months of completion of primary carboplatin/paclitaxel chemotherapy. By logistic regression with multiple simulations using leave one out cross validation, the inventors have identified a 5 protein signature (src(p)Tyr416 (note: X(p)Y designates phosphorylation of protein X at amino acid Y), AKT, HER2, S6(p)Ser235/236 and CCND1 (cyclin Dl)) with a sensitivity, specificity, positive, and negative predictive value of 81%, 94%, 78% and 87%, respectively, for prediction of primary ‘platinum resistance’. Using committee modeling developed by Dr. Jonas Almeida/Wenbin Liu (Dept. of Bioinformatics and Computational Biology) (unpublished)), the inventors have further refined the analysis of protein signaling patterns associated with primary ovarian cancer ‘platinum resistance’ by identification of unique individual tumor functional proteomic signatures. Results of this study demonstrates markedly different components of the functional proteomic ‘fingerprints’ from ovarian cancers in patient with progression-free survivals (PFS) of 0.66 months and 21 months after completion of primary chemotherapy. This approach identifies prominent protein signaling ‘fingerprints’ in individual ovarian cancers and remarkably demonstrates: (1) significant concordance in tumors from patients with progression-free survivals (PFS) of 6 months or less after primary carboplatin-based therapy (i.e., with primary ‘platinum resistant’ ovarian cancers), (2) overlap with the primary ‘platinum resistance’ model identified using logistic regression, with similarity in the major protein components of the signatures, including src and AKT, and (3) Receiver Operator Characteristic (ROC) curves with excellent sensitivities/specificities (AUCs>90%). Based on the sensitivity and specificity of the committee modeling approach for prediction of PFS in individual ovarian cancer patients after completion of standard primary chemotherapy, the inventors have incorporated this into software that will be used for analysis of RPPA data to be derived from validation ovarian cancer sets to be analyzed. Of note, other modeling methodology, such as Xcluster and Treeview, can be used to arrive at similar results.

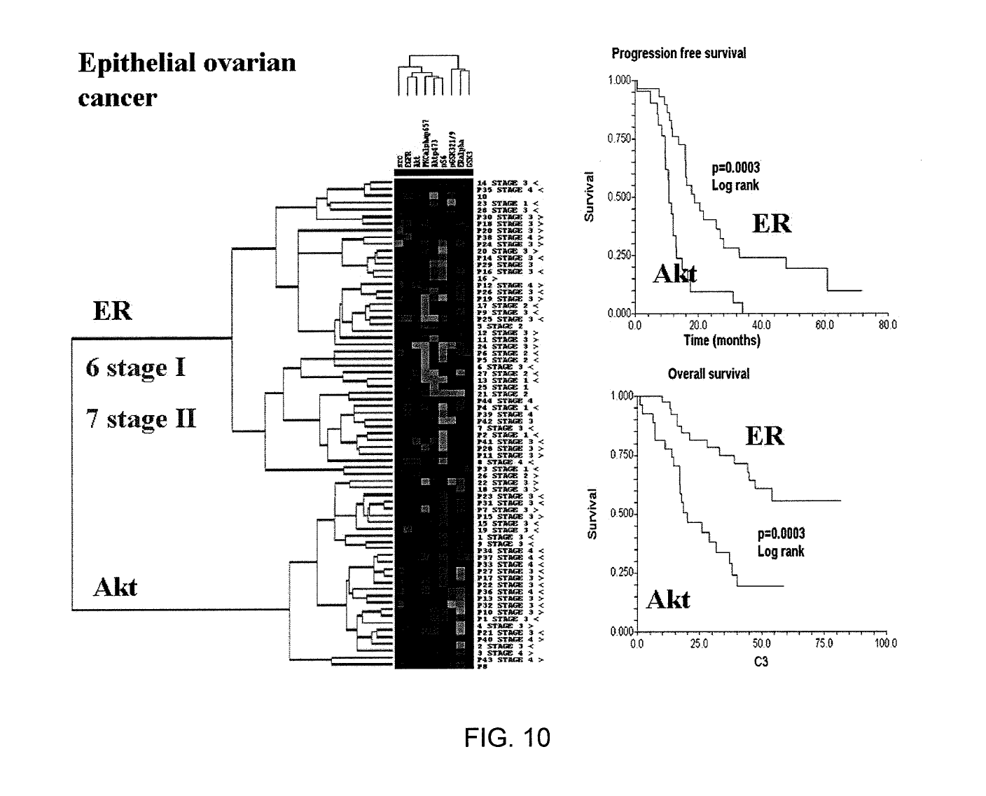

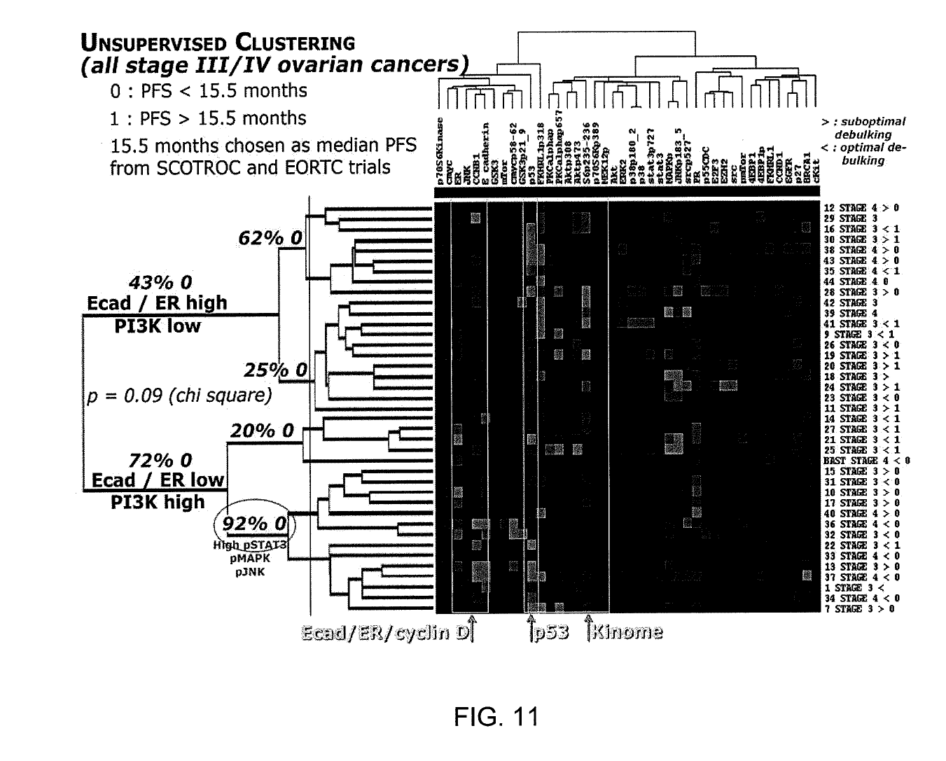

Using unsupervised and supervised clustering with softwares including Xcluster and Treeview, other potentially powerful prognostic and predictive signatures have also been trained and developed in patients with ovarian cancer (FIG. 10). Even unsupervised approaches can distinguish ovarian cancer tumor subsets with significantly different survival outcomes (FIG. 11). These signatures will have clinical utility in guiding the management and treatment of ovarian cancer patients.

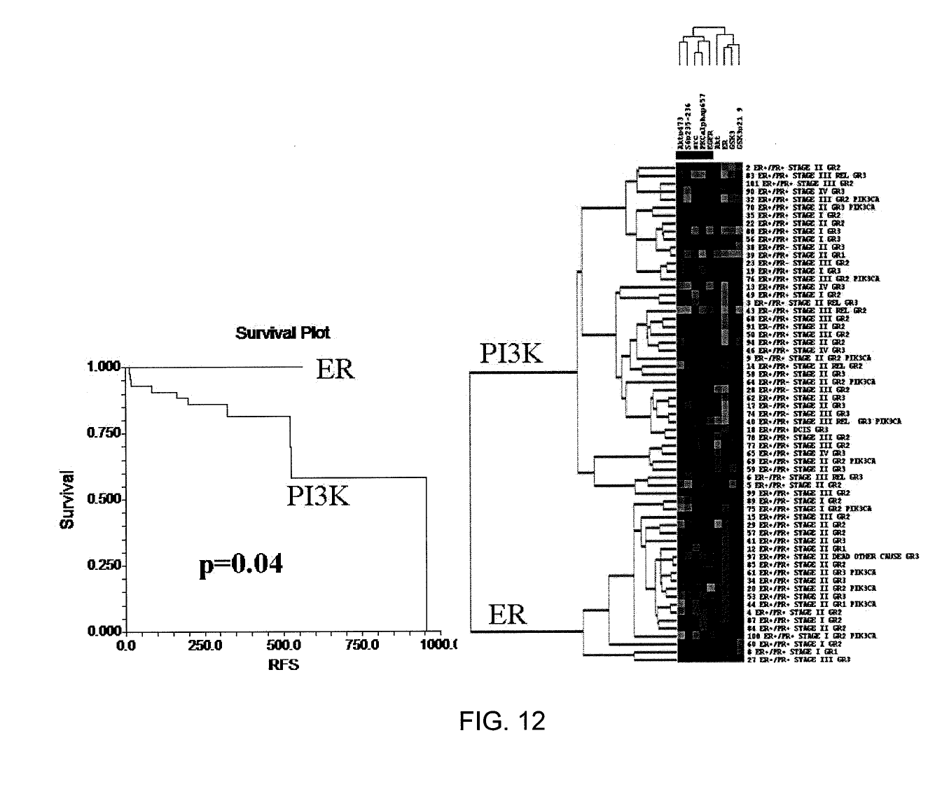

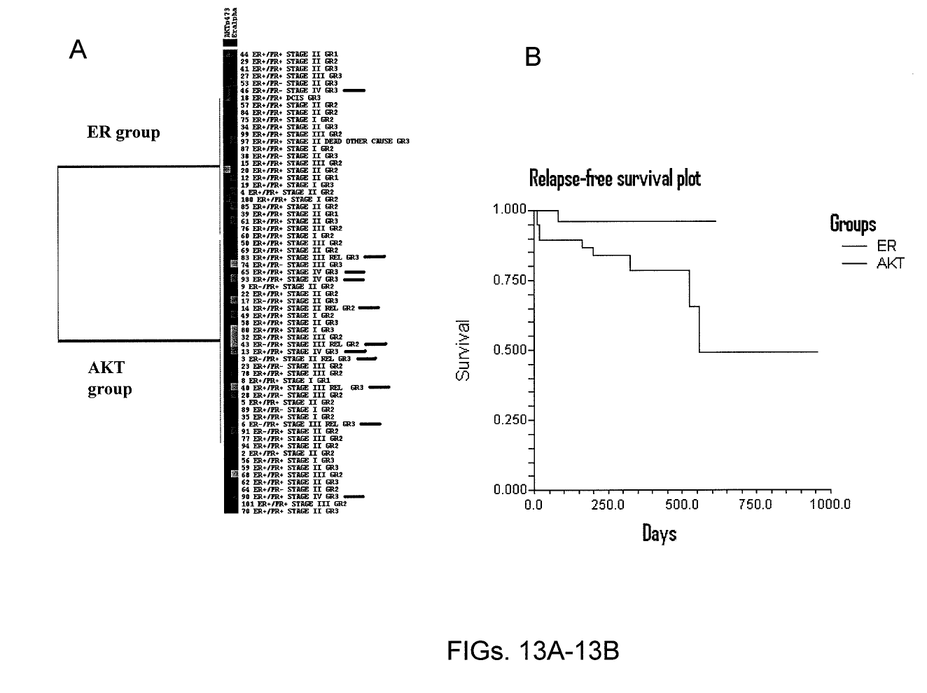

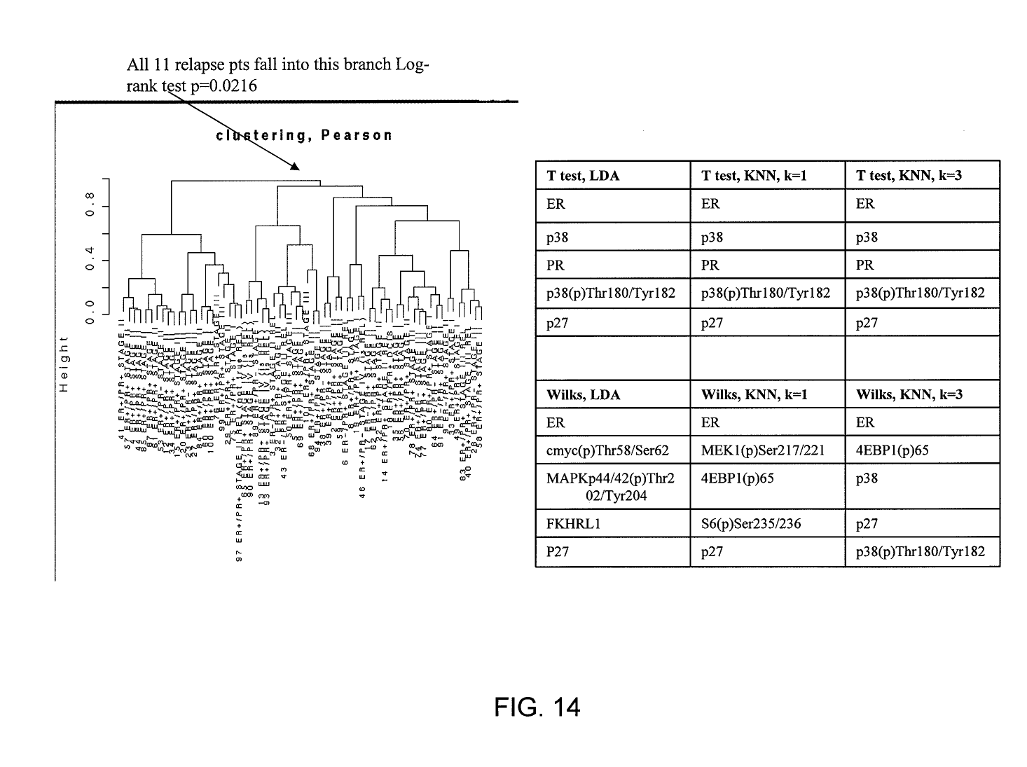

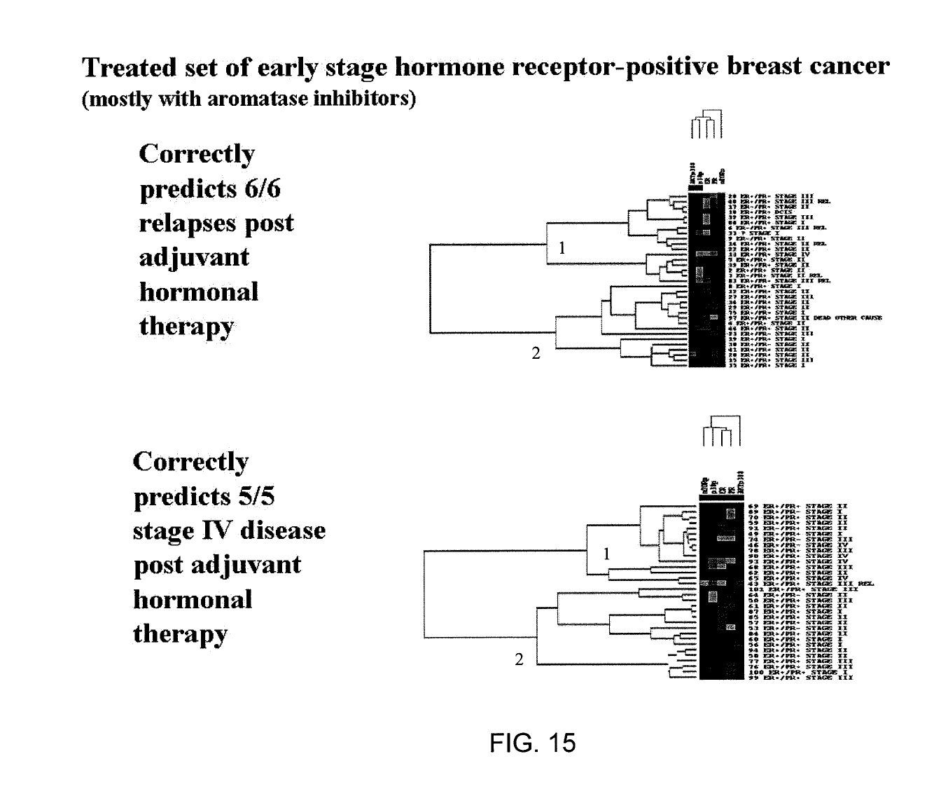

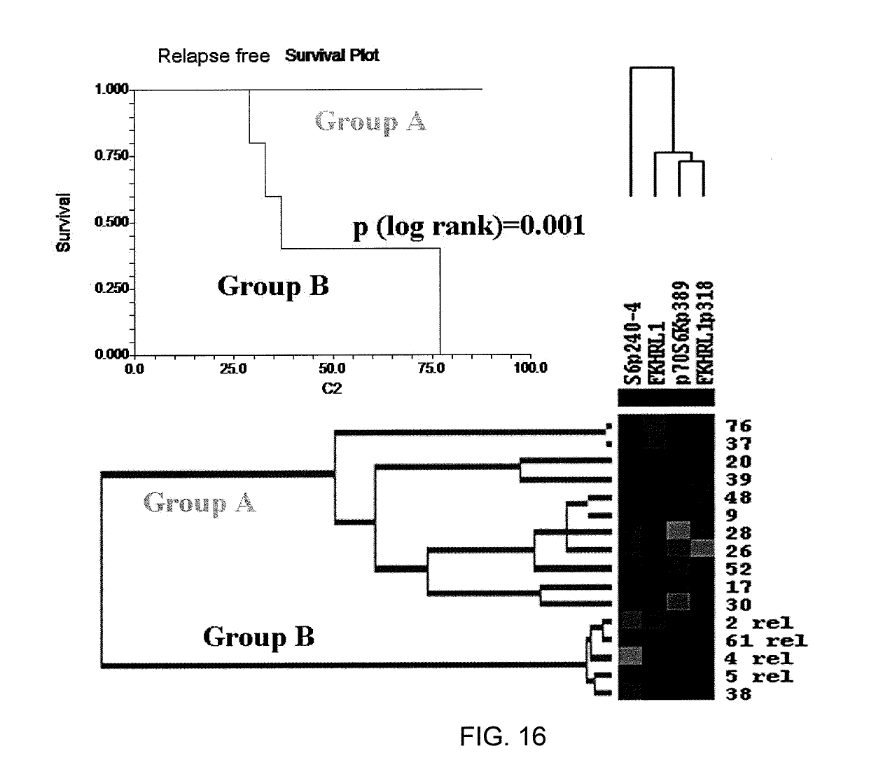

Antihormone treated breast cancer. In antihormone-treated early stage hormone receptor-positive breast cancer, utilizing RPPA with antibodies shown in Table 1, the inventors have demonstrated in 65 early stage hormone receptor-positive breast cancer patients treated with adjuvant hormonal therapy that there are significant inverse correlations between activation of intracellular kinase pathway components and the level of tumor expression of hormone receptors. Signatures derived using RPPA data to reflect this inverse relationship are significantly predictive of outcome in these treated patients (FIG. 12). This is similar to preliminary data above in ovarian cancer. However, unlike in ovarian cancer, when only ER expression and Akt phosphorylation are used, the breast cancer signature retains significant predictive capability after adjuvant antihormone therapy. This is shown in FIG. 13 utilizing AKT phosphorylation at Serine 473 (AKT(p)Ser473 as a surrogate for PI3K pathway activation) and ERα level (p=0.02 for significant inverse correlation). This signature of low (typically green in heat map data display) ERα expression with high (typically red in heat map data display) AKT(p)Ser473 also provides strong prediction of disease recurrence after adjuvant antihormone therapy (relapses marked by black line in FIG. 13A). FIG. 14 shows an alternative approach to analysis of breast cancer RPPA data by resampling analysis using pearson correlation, linear discriminant analysis (LDA) and K nearest neighbors (KNN) methodology to determine (phospho)proteins most associated with breast cancer relapse after adjuvant antihormone therapy. Supervised clustering of RPPA data reflecting quantitation of the expression/activation of the proteins shown in Table 1 also identifies other predictive biomarker signatures of breast cancer relapse (FIG. 15 and FIG. 16), some have been validated (preliminary validation specifically in antihormone-treated patients with early stage hormone receptor-positive breast cancer).

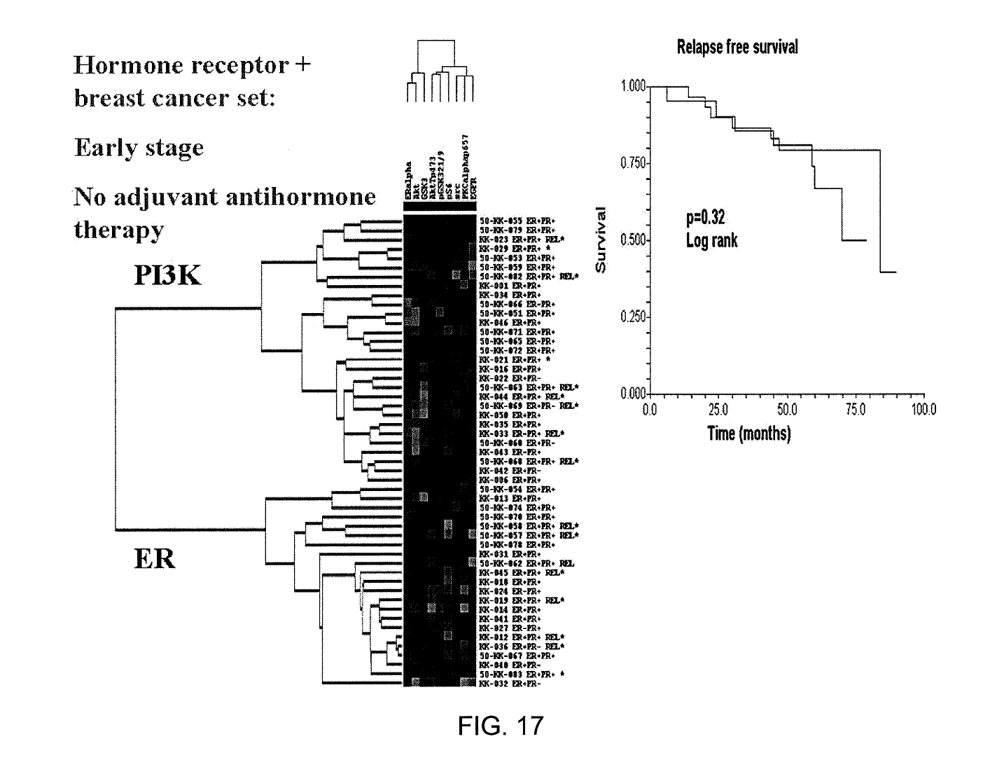

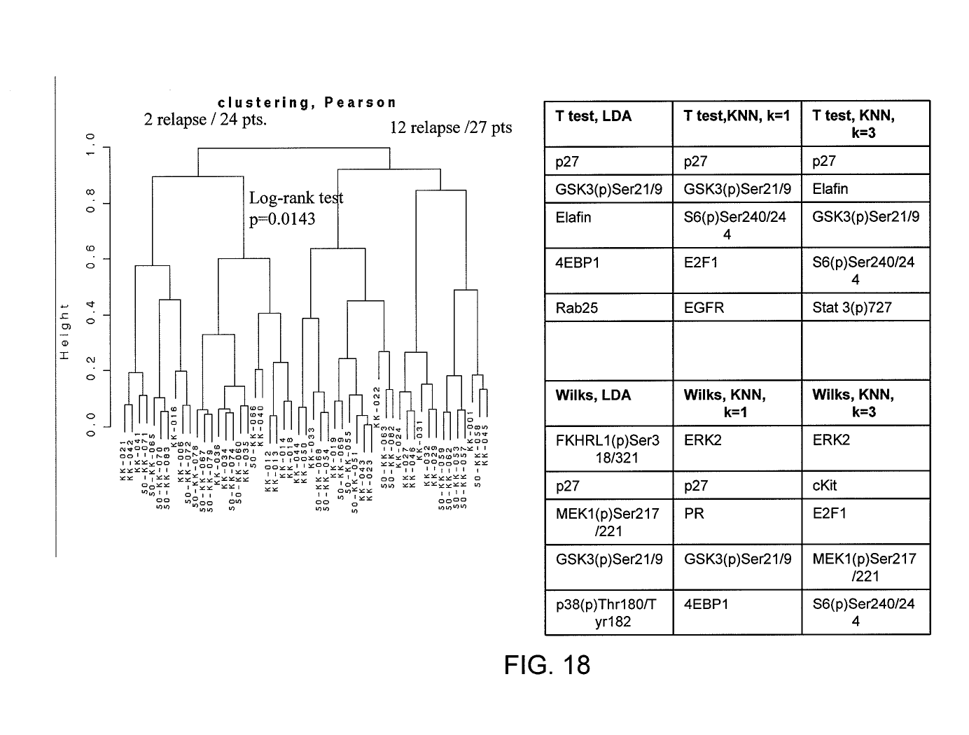

This inverse relationship between kinase and steroid pathway signaling is also reproduced in a primary tumor set derived from 51 early stage hormone receptor-positive untreated breast cancer patients, but in this case the corresponding signatures are not prognostic (i.e., are not predictive of outcome in the absence of adjuvant hormonal therapy treatment—FIG. 17). This suggests that this signature has predictive rather than prognostic utility in early stage hormone receptor-positive breast cancer. FIG. 18 shows an alternative approach to analysis of these RPPA data by resampling analysis using pearson correlation, linear discriminant analysis (LDA) and K nearest neighbors (KNN) methodology to determine (phospho)proteins most associated with early stage hormone receptor-positive breast cancer relapse after no adjuvant antihormone therapy. Clearly, these differ from those (phospho)proteins most associated with breast cancer relapse after adjuvant antihormone therapy (shown in FIG. 14). Hence, the RPPA approach clearly has the capacity to identify differential biomarkers associated with hormone receptor-positive breast cancer relapse in the presence and absence of adjuvant antihormone therapy. Such biomarkers will have utility upon validation in terms of selection of patients for alternative/additional therapy approaches and potentially in determination of treatment targets in these patients.

The inventors have found a proteomic signature of PI3K/AKT/mTOR pathway activation as defined by phosphorylation of AKT, mTOR, GSK3, and p70S6K in over one-third of hormone receptor-positive breast tumors although both sets specifically excluded HER2 amplified breast cancers, providing evidence of frequent but undetermined pathway activation mechanism(s) in hormone receptor-positive breast cancer. Further, these data suggest that kinase signaling interruption may have therapeutic utility in some hormone receptor-positive breast cancer patients who have a poor outcome after treatment with adjuvant antihormone therapy alone.

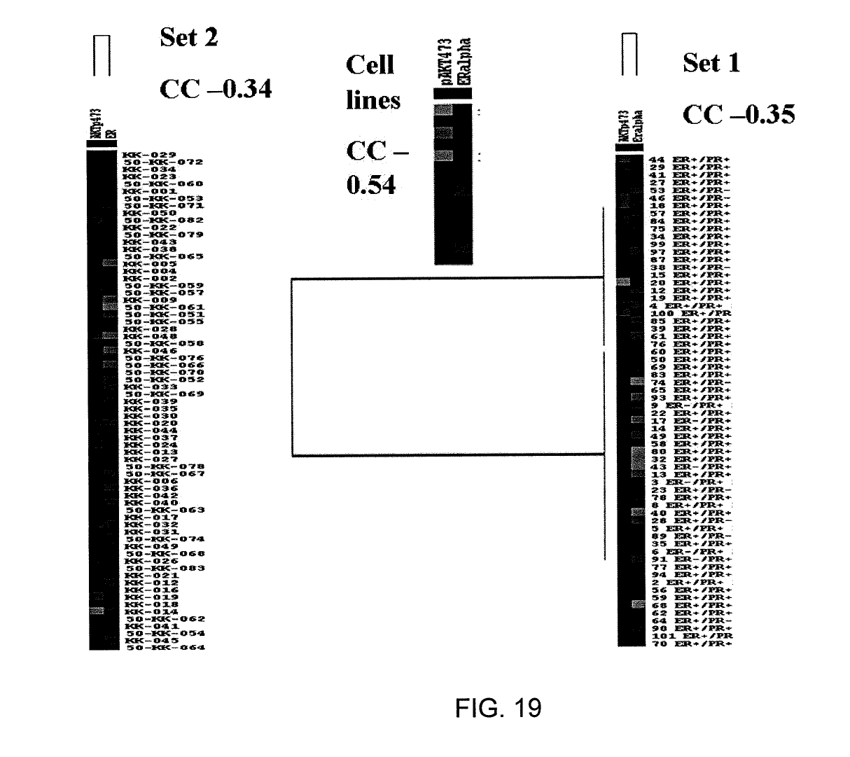

Association between ER expression and PI3K/AKT/mTOR pathway. A striking inverse association between ER expression and PI3K/AKT/mTOR pathway activation has been consistently seen in our breast cancer and ovarian cancer tumor set RPPA data (FIG. 19). This is one example of a potentially important and novel protein-protein association that the RPPA platform is capable of discovering.

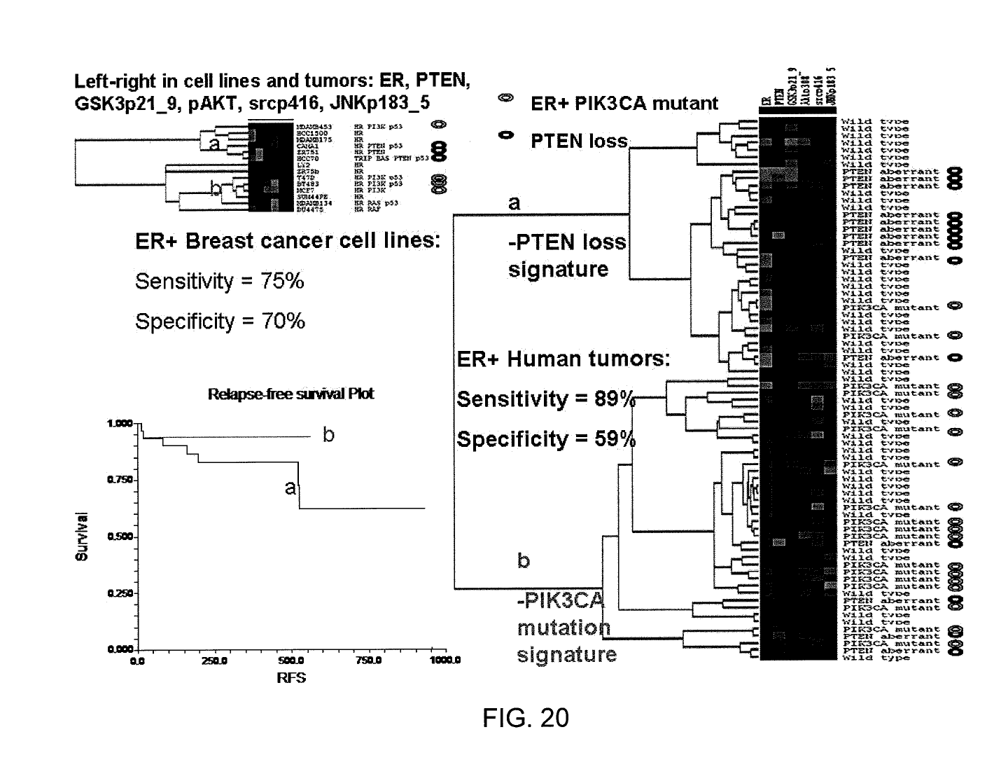

Predictive functional proteomic patterns for PIK3CA. Predictive functional proteomic patterns for PIK3CA mutation and PTEN loss in breast cancer have been derived from the RPPA data and confirmed in two small independent patient sample sets (FIG. 20). These findings require expansion, integration with genomic data, and validation in independent sets of uniformly treated patients with early stage hormone receptor-positive breast cancer but clearly have much potential clinical utility.

TABLE 1
Validated antibodies used in reverse phase protein arrays (RPPA)
to study functional proteomic patterns of relevance to the clinical
behavior of epithelial ovarian and breast cancers. (p) indicates
phosphorylation. Antibodies to the proteins indicated are obtained
from the following companies: Cell Signaling, Inc., Epitomics,
Santa Cruz and BD Pharmingen.

Receptor TKs, steroid, e.g.
EGFR
EGFR(p)Tyr1068
EGFR(p)Tyr1173
ERα, PR, pS2
ERα(p)Ser104/106
ERα(p)Ser118/167
ERα(p)Ser236
ERα(p)Ser305
ERCC1
HER2/HER2(p)Tyr1248
HER3
cKit/IGER1/IRS1
PKCα/PKCα (p)657
src/src(p)Tyr416/527
PI3K/AKT/pathway, e.g.
4EBP1/4EBP1(p)65
AKT
AKT(p)Thr308
AKT(p)Ser473
PI3K/AKT/pathway ctd.
FKHRL1 (FOXO3a)
FKHRL1(p)Ser318/321
FOXO1
FOXO1(p)Ser256
GSK3
GSK3(p)Ser21/9
LKB1
LKB1(p)
mTOR/mTOR(p)Ser2448
p70S6 Kinase
p70S6K(p)Thr389
PI3K subunits p85 and p11 [symbol]
PTEN/PTEN(p) sites
S6 ribosomal protein
S6(p)Ser235/236
S6(p)Ser240/244
MAPK pathway, e.g.
MEK1/2
MEK1(p)Ser217/221
MAPK pathway ctd.
ERK2/MAPKp44/42
(p)Thr202/Tyr204
p38/p38(p)Thr180/Tyr182
JNK
JNK(p)Thr183/Tyr185
Effectors, e.g.
CCNB1/2/CCND1/2
CCNE1/2
cdk2/cdk4
cmyc
cmyc(p)Thr58/
Ser62
E2F1/Elafin
p21/p27/p53/p53(p) sites
PCNA
Rab25
JAK/STAT signaling, e.g.
Stat 3
Stat 3(p)705
JAK/STAT signaling ctd.
Stat 3(p)727
Stat 5/Stat 5(p)
Stat 6/Stat 6(p)694
Apoptosis, e.g.
Bax
Bcl components e.g. Bcl2,
Bcl-xL
Caspase components
Cleaved caspases e.g. 3/7
DR4/DR5
TRAIL/Survivin
From genomic proflling, e.g.
BCMS1/CD133
COMT/connexin 43/EVI1
ETV6/Gelsolin/KRT23
MVP/NOTCH1/PML/PTCH
RBM15/Rho C/ROPN1/SIL
SHO/Telomerase/TPM3
YB1

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,376,110
U.S. Pat. No. 4,816,567
U.S. Patent Publn. 2006/0040338
Adams and Schier, J. Immunol. Methods, 231:249-260, 1999.
Altomare et al., Oncogene 23 (2004), pp. 5853-5857.
Atwell et al., Protein Eng., 12:597-604, 1999.
Ayers et al., J. Clin. Oncol., 22:1-10, 2004.
Bachman et al., Cancer Biol. Ther., 3:772-775, 2004.
Bird et al., Science, 242:423-426, 1988.
Burke et al., Oncogene 20 (2001), pp. 7925-7934.
Charboneau et al., Brief Funct. Genomic Proteomic, 1:305-315, 2002.
Cheng et al., Proc. Natl. Acad. Sci. USA 89 (1992), pp. 9267-9271.
Chu et al., J. Clin. Invest, 2007 In Press.
Colcher et al., J. Nucl. Med., 42:225-241, 1998.
David et al., Biochemistry, 13:1014, 1974.
Diks and Peppelenbosch, Trends Mol. Med., 10:574-577, 2004.
Early breast Cancer Trialists, In: Polychemotherapy for early breast cancer: an overview of the randomized trials, Early Breast Cancer Trialists' Collaborative Group, Lancet, 352:930-942, 1998.
Fisher et al., Cancer, 95:681-695, 2002.
Goding, In: Monoclonal Antibodies: Principles and Practice, 2d ed., Academic Press, Orlando, Fla., pp 60-61, 71-74, 1986.
Grubb et al., Proteomics, 3:2142-2146, 2003.
Gygi et al., Mol. Cell Biol., 19:1720-1730, 1999.
Hennessy and Mills, Ovarian cancer: Linking Genomics to New Target Discovery and Molecular Markers: The Way Ahead. In Hormonal Carcinogenesis. Springer Verlag, 2007 In press.
Hennessy et al., Nat. Rev. Drug Discov. 4 (2005), pp. 988-1004.
Hennessy et al., Submitted 2007.
Hunter et al., Nature, 144:945, 1962.
Huston et al., Biochemistry, 27 (25):8945-8952, 1988.
Jones et al., Nature, 321:522-525, 1986.
Kerr and Churchill, Proc. Natl. Acad. Sci. USA, 98 (16):8961-8965, 2001.
Kohler and Milstein, Nature, 256:495-497, 1975.
Lancaster et al, Clin. Cancer Res. 9 (2003), pp. 762-766.
Lev et al., Br. J. Cancer, 91:795-802, 2004.
Levine et al., Clin. Cancer Res. 11 (2005), pp. 2875-287.
Liang et al., Nat. Cell Biol. 9 (2006), pp. 218-224.
Mandell, Amer. J. Pathology, 163:1687-1698, 2003.
Morrison et al., Proc. Natl. Acad. Sci. USA, 81 (21):6851-6855, 1984.
Munson and Pollard, Anal. Biochem., 107:220, 1980.
Murthy et al., Arch. Pathol. Lab. Med., 129:39-46, 2005.
Nygren, J. Histochem. Cytochem., 30 (5):407-412, 1982.
Paik et al., N. Engl. J. Med., 351:2817-2826, 2004
Pain et al., J. Immunol. Meth., 40:219, 1981.
Parkin et al., Int. J. Cancer, 94:153-156, 2000.
Pisani et al., Int. J. Cancer, 97:72-81, 2002.
Pusztai et al., San Antonio, Tex., Breast Cancer Conference, Abst. 112, 2004.
Real et al, Oncogene 21 (2002), pp. 7611-7618.
Riechmann et al., Nature, 332 (6162):323-327, 1988.
Shayesteh et al., Nat. Genet. 21 (1999), pp. 99-102.
Silva et al., Mol Endocrinology 2007; epub ahead of print.
Sorlie et al., Proc. Natl. Acad. Sci. USA, 98:10869-10874, 2001.
Stoica et al., Oncogene, 22:7998-8011, 2003.
Tibes et al., Mol. Can. Ther. 5 (2006), pp. 2512-2521.
Verhoeyen et al., Science, 239 (4847): 1534-1536, 1988.
Watson, Expert. Rev. Mol. Med. 8 (2006), pp. 1-15.
Wulfkuhle et al., Proteomics 2003; 3:2085-2090, 2003.
Zhang et al., Cancer Res. 63 (2003), pp. 4225-4231.
Zola, In: Monoclonal Antibodies: A Manual of Techniques, CRC Press, Inc., pp.147-158, 1987.

What is claimed is:

1. A method for evaluating a cancer patient for propensity to respond to a therapy comprising:
(a) contacting a sample comprising cancer cell proteins from the cancer patient with at least two antibodies under binding conditions, wherein the antibodies are antibodies that bind E cadherin, 4EBP, protein kinase C (PKC), p53, estrogen receptor (ER), progesterone receptor (PR), S6, AKT, Her2, Src, PI3K, p38, p27, mTOR, c-jun N-terminal kinase (JNK), MAPK (44/42), cyclin D1, or cyclin B 1;
(b) analyzing the binding of the antibodies to the proteins to generate an antibody binding profile;
(c) comparing the antibody binding profile to:
(i) an antibody binding profile indicative of a patient that responds to a therapy, and/or
(ii) an antibody binding profile indicative of a patient that does not respond to a therapy; and
(d) evaluating the cancer patient's propensity for response to the therapy.

2. The method of claim 1, wherein the cancer cell protein is contacted with at least three, at least four, at least five or at least twenty different antibodies.

3. The method of claim 1, wherein at least one antibody binds a hormone receptor.

4. The method of claim 3, wherein the hormone receptor is estrogen receptor or progesterone receptor.

5. The method of claim 1, wherein at least one antibody binds a kinase.

6. The method of claim 5, wherein the kinase is Akt, p38, mTor, PI3K, MAPK, JNK or S6.

7. The method of claim 5, wherein the kinase binding antibody is a phosphorylation specific antibody.

8. The method of claim 1, wherein at least one antibody binds to a protein in the Her2, PI3K, MAPK or STAT pathway.

9. The method of claim 1, wherein the antibodies bind at least ER and p38.

10. The method of claim 9, wherein the antibodies bind at least ER, PR, AKT, p38, and mTOR.

11. The method of claim 1, wherein the antibodies bind at least two of ER, E cadherin, AKT, MAPK (44/42), C-jun N-Terminal kinase (JNK), or S6.

12. The method of claim 1, wherein the antibodies bind at least ER, E cadherin, AKT, MAPK (44/42), C-jun N-Terminal kinase (JNK), and S6.

13. The method of claim 1, wherein the antibodies bind at least src, AKT, HER2, S6, and cyclin D1.

14. The method of claim 1, wherein the cancer patient is a lung, breast, brain, prostate, spleen, pancreatic, cervical, ovarian, head and neck, esophageal, liver, skin, kidney, leukemia, bone, testicular, colon, or bladder cancer patient.

15. The method of claim 14, wherein the cancer patient is a breast or ovarian cancer patient.

16. The method of claim 14, wherein the cancer patient is a breast cancer patient and the antibody panel comprises antibodies that bind to estrogen receptor and phosphorylated p38.

17. The method of claim 14, wherein the cancer patient is an ovarian cancer patient and the antibody panel comprises antibodies that bind to estrogen receptor, E cadherin, phosphorylated Akt, phosphorylated MAPK, phosphorylated JNK and phosphorylated S6.

18. The method of claim 1, wherein the therapy is a chemotherapy, a radiation therapy, an immunotherapy, or a surgical therapy.

19. The method of claim 18, wherein the therapy is a chemotherapy.

20. The method of claim 19, wherein the chemotherapy is a cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, paclitaxel, gemcitabien, navelbine, farnesyl-protein transferase inhibitors, transplatinum, 5-fluorouracil, vincristin, Velcade, vinblastin or methotrexate therapy.

21. The method of claim 1, analyzing the binding of antibodies is by quantifying the binding of the antibodies.

22. The method of claim 21, wherein quantifying the binding of the antibodies to the proteins is used to determine the concentration or post-translational modification of a protein.

23. The method of claim 21, wherein quantifying the binding of the antibodies to the proteins is used to determine the concentration of an activated protein.

24. The method of claim 1, further comprising the step of treating cells of a patient with an composition that inhibits or stimulates cell proliferation prior to step (a).

25. The method of claim 24, wherein treating is in vitro.

26. The method of claim 24, wherein the composition comprises a hormone or a growth factor.

27. The method of claim 24, wherein the composition comprises a kinase inhibitor or a chemotherapeutic agent.

28. The method of claim 1, wherein the method is performed on a microarray.

29. A kit for predicting a cancer patient's response to a therapy comprising one or more of a panel of antibodies, a composition for detecting antibody binding to proteins, one or more reference antibody binding profile, a microarray slide, a protein extraction buffer, a cell proliferation inhibitor, a cell proliferate stimulator, or a computer program for comparing antibody binding profiles.
